(19) United States
(12) Patent Application Publication
(10) Pub. No.: US 2025/0380851 A1
(43) Pub. Date: Dec. 18, 2025

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Rito MURASE, Kanagawa (JP)

(73) Assignee: FUJIFILM Corporation, Tokyo (JP)

(21) Appl. No.: 19/315,702

(22) Filed: Sep. 1, 2025

Related U.S. Application Data
(63) Continuation of application No. PCT/JP2024/003505, filed on Feb. 2, 2024.

(30) Foreign Application Priority Data
Mar. 7, 2023 (JP) 2023-034904

(51) Int. Cl. A61B 1/00; G06T 7/00; G06T 7/60; G06V 10/25; G06V 10/26; G06V 10/70; G06V 20/40; G06V 20/50; G16H 30/40; G16H 40/63

(52) U.S. Cl. CPC A61B 1/0005; A61B 1/000096; G06T 7/0016; G06T 7/60; G06V 10/25; G06V 10/70; G06V 20/40; G06V 20/50; G16H 30/40; G16H 40/63; G06T 2207/10016; G06T 2207/10068; G06T 2207/30096; G06V 10/26; G06V 2201/03

(57) ABSTRACT

A medical support device includes a processor. The processor is configured to: acquire size-related information that is information corresponding to sizes in a time series of an observation target region shown in a medical video image; and output the size-related information. A representative value of the sizes in the time series is used as the size-related information.

### CROSS-REFERENCE TO RELATED APPLICATIONS

This application is a continuation application of International Application No. PCT/JP2024/003505, filed Feb. 2, 2024, the disclosure of which is incorporated herein by reference in its entirety. Further, this application claims priority from Japanese Patent Application No. 2023-034904, filed Mar. 7, 2023, the disclosure of which is incorporated herein by reference in its entirety.

### BACKGROUND

### 1. Technical Field

The technology of the present disclosure relates to a medical support device, an endoscope system, a medical support method, and a program.

### 2. Related Art

JP2015-167629A discloses a medical image processing device including an image storage unit, an image acquisition unit, a reference point setting unit, a part measurement unit, a change amount calculation unit, an annotation and graph generation unit, and a display unit.

In the medical image processing device disclosed in JP2015-167629A, the image storage unit chronologically stores a plurality of examination images having different imaging dates and times for each patient. The image acquisition unit acquires the examination images from the image storage unit. The reference point setting unit sets a reference point based on a region of interest in the examination image. The part measurement unit acquires a measured value of a measurement item of the region of interest in any direction with the reference point as a center. A change amount calculation unit calculates a time-series change amount of the measured value. The annotation and graph generation unit generates an annotation and a graph representing a change amount during a follow-up observation period. The display unit displays the annotation and the graph on a screen.

JP2015-066129A discloses a fluorescence observation device comprising a signal light source, an excitation light source, an image sensor, an oxygen saturation calculation unit, a reference region setting unit, a region-of-interest setting unit, a standardized fluorescence intensity calculation unit, a fluorescence image generation unit, and a display unit.

In the fluorescence observation device disclosed in JP2015-066129A, the signal light source irradiates a sample with signal light having a wavelength range in which a fluorescence coefficient changes in accordance with oxygen saturation of hemoglobin in blood. The excitation light source irradiates the sample with excitation light for exciting a fluorescent substance contained in the sample to emit fluorescence. The image sensor images the sample with the signal light to output a first image signal, and images the sample with the fluorescence to output a second image signal.

In the fluorescence observation device disclosed in JP2015-066129A, the oxygen saturation calculation unit calculates, for each pixel, the oxygen saturation of the sample based on the first image signal. The reference region setting unit sets a reference region of the sample based on the oxygen saturation. The region-of-interest setting unit sets a region of interest of the sample. The standardized fluorescence intensity calculation unit calculates a standardized fluorescence intensity representing a standardized luminescence intensity of the fluorescence by dividing a region-of-interest fluorescence intensity calculated using a pixel value of a region of interest of the second image signal by a reference fluorescence intensity calculated using a pixel value of a reference region of the second image signal. The fluorescence image generation unit generates a fluorescence image in which the region of interest is pseudo-colored based on the standardized fluorescence intensity. The display unit displays, in time series, a plurality of fluorescence images obtained by imaging the same sample at two or more different times.

JP2020-514851A discloses a tumor tracking device including a guideline engine including one or a plurality of processors, a detection engine including one or a plurality of processors, and a user interface. In the tumor tracking device disclosed in JP2020-514851A, the processor of the guideline engine receives a current measured value and a plurality of previous measured values of at least one lesion based on a target medical image, and each of the current measured value and the plurality of previous measured values is identified in time series, and the processor of the guideline engine calculates growth between the current measured value and a latest measured value among the plurality of previous measured values.

The processor of the detection engine calculates growth between the current measured value and each of non-latest measured values among the plurality of previous measured values. The detection engine identifies at least one non-latest measured value based on the results in which the calculated growth between the current measured value and each of the plurality of non-latest measured values among the plurality of previous measured values exceeds a threshold value set by a medical guideline and the calculated growth between the current measured value and the latest measured value among the plurality of previous measured values does not exceed the threshold value. The user interface includes one or a plurality of processors that display an indicator of at least one identified non-latest measured value of at least one lesion on a display device.

### SUMMARY

One embodiment according to the technology of the present disclosure provides a medical support device, an endoscope system, a medical support method, and a program that enable a user or the like to accurately ascertain a size of an observation target region shown in a medical video image.

A first aspect according to the technology of the present disclosure relates to a medical support device comprising: a processor, in which the processor is configured to: acquire size-related information that is information corresponding to sizes in a time series of an observation target region shown in a medical video image; and output the size-related information, and a representative value of the sizes in the time series is used as the size-related information.

A second aspect according to the technology of the present disclosure relates to the medical support device according to the first aspect, in which the representative value is a value that is representative of the sizes measured in the time series based on a plurality of frames included in a first period of the medical video image.

A third aspect according to the technology of the present disclosure relates to the medical support device according to the second aspect, in which the representative value includes a maximum value of the sizes within the first period, a minimum value of the sizes within the first period, a frequency of the sizes within the first period, an average value of the sizes within the first period, a median value of the sizes within the first period, and/or a variance value of the sizes within the first period.

A fourth aspect according to the technology of the present disclosure relates to the medical support device according to the second or third aspect, in which the representative value includes a frequency of the sizes within the first period, and a histogram of the frequency is used as the size-related information.

A fifth aspect according to the technology of the present disclosure relates to the medical support device according to any one of the second to fourth aspects, in which the representative value includes a maximum value of the size and a minimum value within the first period, and fluctuation range information indicating a fluctuation range from the maximum value to the minimum value is used as the size-related information.

A sixth aspect according to the technology of the present disclosure relates to the medical support device according to any one of the first to fifth aspects, in which the processor is configured to acquire the size-related information in a case in which the sizes in the time series are stable.

A seventh aspect according to the technology of the present disclosure relates to the medical support device according to the sixth aspect, in which the processor is configured to: output the size-related information in a case in which the sizes in the time series are stable; and not output the size-related information in a case in which the sizes in the time series are not stable.

An eighth aspect according to the technology of the present disclosure relates to the medical support device according to the sixth or seventh aspect, in which the processor is configured to: output the sizes in a case in which the sizes in the time series are stable; and not output the sizes in a case in which the sizes in the time series are not stable.

A ninth aspect according to the technology of the present disclosure relates to the medical support device according to any one of the sixth to eighth aspects, in which whether or not the sizes in the time series are stable is determined based on a recognition result of the observation target region, measurement results of the sizes, and/or an appearance of the observation target region shown in the medical video image.

A tenth aspect according to the technology of the present disclosure relates to the medical support device according to the ninth aspect, in which, in a case in which a change amount of the sizes in the time series within a second period and/or a change amount of distance information included in a distance image for the observation target region is less than a threshold value, it is determined that the sizes in the time series are stable.

An eleventh aspect according to the technology of the present disclosure relates to the medical support device according to the tenth aspect, in which the observation target region is recognized by a method using AI, the change amount of the sizes is a change amount of a closed region that defines the observation target region recognized by the method using the AI, the closed region is a bounding box or a segmentation image obtained from the AI, and the change amount of the distance information is a change amount of the distance information included in the distance image corresponding to the closed region.

A twelfth aspect according to the technology of the present disclosure relates to the medical support device according to any one of the ninth to eleventh aspects, in which the appearance includes a blurriness amount, a shake amount, brightness, an angle of view, a position, and/or a direction.

A thirteenth aspect according to the technology of the present disclosure relates to the medical support device according to any one of the ninth to twelfth aspects, in which the processor is configured to output determination result information indicating whether or not the sizes in the time series are stable.

A fourteenth aspect according to the technology of the present disclosure relates to the medical support device according to any one of the first to thirteenth aspects, in which the output of the size-related information is implemented by displaying the size-related information on a first screen.

A fifteenth aspect according to the technology of the present disclosure relates to the medical support device according to the fourteenth aspect, in which the processor is configured to: selectively display, on the first screen, temporal change information that enables specification of a temporal change of the sizes in the time series and the size-related information; and in a case in which the sizes in the time series are stable in a state in which the temporal change information is displayed on the first screen, switch information displayed on the first screen from the temporal change information to the size-related information.

A sixteenth aspect according to the technology of the present disclosure relates to the medical support device according to the fourteenth or fifteenth aspect, in which the processor is configured to change a display aspect of the size-related information on the first screen in accordance with whether or not the sizes in the time series are stable.

A seventeenth aspect according to the technology of the present disclosure relates to the medical support device according to any one of the first to sixteenth aspects, in which the processor is configured to: display the sizes in the time series on a second screen; and change a display aspect of the sizes on the second screen in accordance with whether or not the sizes in the time series are stable.

An eighteenth aspect according to the technology of the present disclosure relates to the medical support device according to any one of the first to seventeenth aspects, in which the processor is configured to display the sizes in the time series on a third screen, the size displayed on the third screen is a real number represented by a plurality of digits, and a font size, a font color, and/or font brightness of the real number is changed in units of the digits.

A nineteenth aspect according to the technology of the present disclosure relates to the medical support device according to any one of the first to eighteenth aspects, in which the processor is configured to: display a recognition result of the observation target region and/or measurement results of the sizes in a state of being superimposed on the medical video image; and display the size-related information in a separate display region from the medical video image.

A twentieth aspect according to the technology of the present disclosure relates to the medical support device according to any one of the first to nineteenth aspects, in which the medical video image is an endoscopic video image obtained by imaging with an endoscope.

A twenty-first aspect according to the technology of the present disclosure relates to the medical support device according to any one of the first to twentieth aspects, in which the observation target region is a lesion.

A twenty-second aspect according to the technology of the present disclosure relates to an endoscope system comprising: the medical support device according to any one of the first to twenty-first aspects; and an endoscope that is inserted into a body including the observation target region and that images the observation target region to acquire the medical video image.

A twenty-third aspect according to the technology of the present disclosure relates to a medical support method comprising: acquiring size-related information that is information corresponding to sizes in a time series of an observation target region shown in a medical video image; and outputting the size-related information, in which a representative value of the sizes in the time series is used as the size-related information.

A twenty-fourth aspect according to the technology of the present disclosure relates to the medical support method according to the twenty-third aspect, further comprising: using an endoscope that performs imaging to acquire the medical video image.

A twenty-fifth aspect according to the technology of the present disclosure relates to a program causing a computer to execute medical support processing comprising: acquiring size-related information that is information corresponding to sizes in a time series of an observation target region shown in a medical video image; and outputting the size-related information, in which a representative value of the sizes in the time series is used as the size-related information.

### BRIEF DESCRIPTION OF THE DRAWINGS

Exemplary embodiments according to the technique of the present disclosure will be described in detail based on the following figures, wherein:

FIG. 1 is a conceptual diagram showing an example of an aspect in which an endoscope system is used;

FIG. 2 is a conceptual diagram showing an example of an overall configuration of the endoscope system;

FIG. 3 is a block diagram showing an example of a hardware configuration of an electrical system of the endoscope system;

FIG. 4 is a block diagram showing an example of a main function of a processor included in the endoscope system according to the embodiment and an example of information according to the embodiment, which is stored in an NVM;

FIG. 5 is a conceptual diagram showing an example of processing contents of a recognition unit and a control unit according to the embodiment;

FIG. 6 is a conceptual diagram showing an example of a processing content of a measurement unit;

FIG. 7 is a conceptual diagram showing an example of a processing content of a determination unit;

FIG. 8 is a conceptual diagram showing an example of a processing content of an acquisition unit;

FIG. 9 is a conceptual diagram showing an example of an aspect in which an endoscopic video image and a size are displayed in a first display region and size-related information is displayed in a second display region;

FIG. 10 is a flowchart showing an example of a flow of medical support processing;

FIG. 11 is a conceptual diagram showing a modification example of a display aspect of the sizes displayed in the first display region;

FIG. 12 is a conceptual diagram showing an example of an aspect in which the determination unit determines whether or not sizes in a time series are stable using a change amount of the sizes of a segmentation image;

FIG. 13 is a conceptual diagram showing an example of an aspect in which display of temporal change information and display of the size-related information are switched;

FIG. 14 is a conceptual diagram showing an example of an aspect in which display of the temporal change information and display of a histogram are switched;

FIG. 15 is a conceptual diagram showing an example of an aspect in which display of the temporal change information and display of a box-and-whisker plot are switched;

FIG. 16 is a conceptual diagram showing an example of an aspect in which determination result information is displayed on a screen;

FIG. 17 is a conceptual diagram showing a first modification example of display contents of the screen in a case in which the determination unit determines that the sizes in the time series are not stable;

FIG. 18 is a conceptual diagram showing a first modification example of display contents of the screen in a case in which the determination unit determines that the sizes in the time series are stable;

FIG. 19 is a conceptual diagram showing a second modification example of the display contents of the screen in a case in which the determination unit determines that the sizes in the time series are not stable;

FIG. 20 is a conceptual diagram showing a third modification example of the display contents of the screen in a case in which the determination unit determines that the sizes in the time series are not stable;

FIG. 21 is a conceptual diagram showing an example of a processing content of the determination unit in a case in which the determination of whether or not the sizes in the time series are stable is performed based on a size change amount and appearance information;

FIG. 22 is a conceptual diagram showing an example of a processing content of the determination unit in a case in which the determination of whether or not the sizes in the time series are stable is performed based on the size change amount, the appearance information, and a recognition result;

FIG. 23 is a conceptual diagram showing an example of processing contents of the measurement unit and the determination unit in a case in which the determination of whether or not the sizes in the time series are stable is performed based on a change amount of distance information;

FIG. 24 is a conceptual diagram showing an example of an output destination of various types of information; and

FIG. 25 is a conceptual diagram showing an example of a series of processing in which a processor of the endoscope system issues a processing execution request to an external device via a network, the external device executes processing in response to the processing execution request, and the processor of the endoscope system receives a processing result from the external device.

### DETAILED DESCRIPTION

Hereinafter, examples of embodiments of a medical support device, an endoscope system, a medical support method, and a program according to the technology of the present disclosure will be described with reference to the accompanying drawings.

First, the terms used in the following description will be described.

CPU is an abbreviation for “central processing unit”. GPU is an abbreviation for “graphics processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. I/F is an abbreviation for “interface”. SSL is an abbreviation for “sessile serrated lesion”. FIFO is an abbreviation for “first in, first out”.

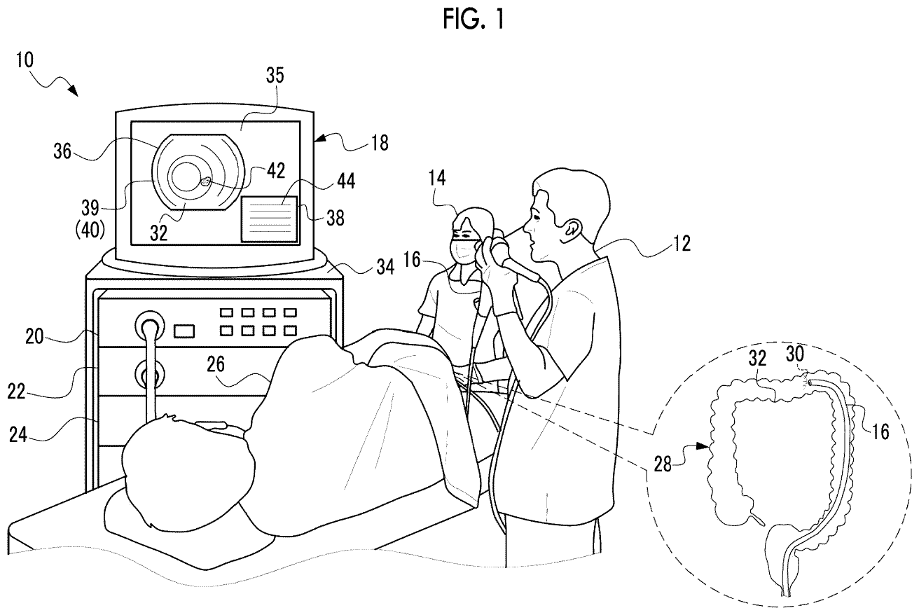

As shown in FIG. 1 as an example, an endoscope system 10 is used by a doctor 12 in endoscopy. A staff member such as a nurse 14 assists with the endoscopy. The endoscope system 10 in the present embodiment is an example of an “endoscope system” according to the technology of the present disclosure.

The endoscope system 10 is connected to a communication device (not shown) communicably, and information obtained by the endoscope system 10 is transmitted to the communication device. Examples of the communication device include a server and/or a client terminal (for example, a personal computer and/or a tablet terminal) that manage various types of information, such as an electronic medical record. The communication device receives the information transmitted from the endoscope system 10, and executes processing using the received information (for example, processing of storing the information in the electronic medical record or the like).

The endoscope system 10 comprises an endoscope 16, a display device 18, a light source device 20, a control device 22, and a medical support device 24. In addition, the endoscope 16 in the present embodiment is an example of an “endoscope” according to the technology of the present disclosure.

The endoscope system 10 is a device for performing medical care for a large intestine 28 included in a body of a subject 26 (for example, a patient), by using the endoscope 16. In the present embodiment, the large intestine 28 is an object to be observed by the doctor 12.

The endoscope 16 is used by the doctor 12, and is inserted into a body cavity of the subject 26. In the present embodiment, the endoscope 16 is inserted into the large intestine 28 of the subject 26. The endoscope system 10 images an inside of the large intestine 28 of the subject 26 by using the endoscope 16 inserted into the large intestine 28 of the subject 26, and performs various medical treatments on the large intestine 28 as necessary.

The endoscope system 10 acquires an image showing a state inside the large intestine 28 by imaging the inside of the large intestine 28 of the subject 26, and outputs the acquired image. In the present embodiment, the endoscope system 10 is an endoscope having an optical imaging function of imaging reflected light obtained by being reflected by an intestinal wall 32 of the large intestine 28 by irradiating the inside of the large intestine 28 with light 30.

It should be noted that, here, the endoscopy of the large intestine 28 has been described as an example, but this is merely an example, and the technology of the present disclosure is applicable to an endoscopy of a luminal organ, such as an esophagus, a stomach, a duodenum, or a trachea.

The light source device 20, the control device 22, and the medical support device 24 are installed in a wagon 34. In the wagon 34, a plurality of tables are provided along an up-down direction, and the medical support device 24, the control device 22, and the light source device 20 are installed from a lower table to an upper table. Further, the display device 18 is installed on an uppermost table in the wagon 34.

The control device 22 controls the entire endoscope system 10. The medical support device 24 performs various types of image processing on the image obtained by imaging the intestinal wall 32 with the endoscope 16, under the control of the control device 22.

The display device 18 displays various types of information including the image. Examples of the display device 18 include a liquid-crystal display and an EL display. A tablet terminal equipped with a display may be used instead of the display device 18 or together with the display device 18.

The display device 18 displays a screen 35. A plurality of display regions are included on the screen 35. The plurality of display regions are arranged on the screen 35. In the example shown in FIG. 1, a first display region 36 and a second display region 38 are shown as examples of the plurality of display regions. A size of the first display region 36 is larger than a size of the second display region 38. The first display region 36 is used as a main display region, and the second display region 38 is used as a sub-display region.

An endoscopic video image 39 is displayed in the first display region 36. The endoscopic video image 39 is an image acquired by imaging the intestinal wall 32 with the endoscope 16 in the large intestine 28 of the subject 26. In the example shown in FIG. 1, a video image in which the intestinal wall 32 is captured is shown as an example of the endoscopic video image 39. The endoscopic video image 39 in the present embodiment is an example of a “medical video image” and an “endoscopic video image” according to the technology of the present disclosure. Further, the first display region 36 in the present embodiment is an example of a “second screen” and a “third screen” according to the technology of the present disclosure. Further, the second display region 38 in the present embodiment is an example of a “first screen” and a “separate display region” according to the technology of the present disclosure.

The intestinal wall 32 shown in the endoscopic video image 39 includes a lesion 42 (for example, in the example shown in FIG. 1, one lesion 42) as a region of interest (that is, an observation target region) focused on by the doctor 12, and the doctor 12 can visually recognize an aspect of the intestinal wall 32, including the lesion 42, through the endoscopic video image 39. The lesion 42 in the present embodiment is an example of an “observation target region” and a “lesion” according to the technology of the present disclosure.

The lesion 42 has various types, and examples of the types of the lesion 42 include a neoplastic polyp and a non-neoplastic polyp. Examples of the types of the neoplastic polyp include an adenomatous polyp (for example, SSL). Examples of the types of the non-neoplastic polyp include a hamartomatous polyp, a hyperplastic polyp, and an inflammatory polyp. It should be noted that the types shown here are types assumed in advance as the types of the lesion 42 in a case in which the endoscopy is performed on the large intestine 28, and the types of the lesion may be different depending on the organ on which the endoscopy is performed.

In the present embodiment, for convenience of description, the form example has been described in which one lesion 42 is included in the endoscopic video image 39, but the technology of the present disclosure is not limited to this, and the technology of the present disclosure is established even in a case in which a plurality of lesions 42 are included in the endoscopic video image 39.

In the present embodiment, the lesion 42 is shown, but this is merely an example, and the region of interest (that is, the observation target region) focused on by the doctor 12 may be an organ (for example, a duodenal papilla), a marked region, an artificial treatment tool (for example, an artificial clip), a treated region (for example, a region in which a trace of removal of a polyp or the like remains), or the like.

The image displayed in the first display region 36 is one frame 40 included in a video image including a plurality of frames 40 arranged in time series. That is, the plurality of frames 40 arranged in time series are displayed in the first display region 36 at a predetermined frame rate (for example, several tens of frames/second). The frame 40 in the present embodiment is an example of a “frame” according to the technology of the present disclosure.

Examples of the video image displayed in the first display region 36 include a live-view video image. The live-view video image is merely an example, and the video image may be a video image, such as a post-view video image, that is temporarily stored in a memory or the like and then displayed. In addition, each frame included in a video image for recording stored in the memory or the like may be reproduced and displayed as the endoscopic video image 39 on the screen 35 (for example, in the first display region 36).

On the screen 35, the second display region 38 is adjacent to the first display region 36 and is displayed in the lower right of the front view on the screen 35. The second display region 38 may be displayed at any position as long as the position is within the screen 35 of the display device 18, but is preferably displayed at a position that is comparable with the endoscopic video image 39. Size-related information 44 is displayed in the second display region 38. Details of the size-related information 44 will be described later.

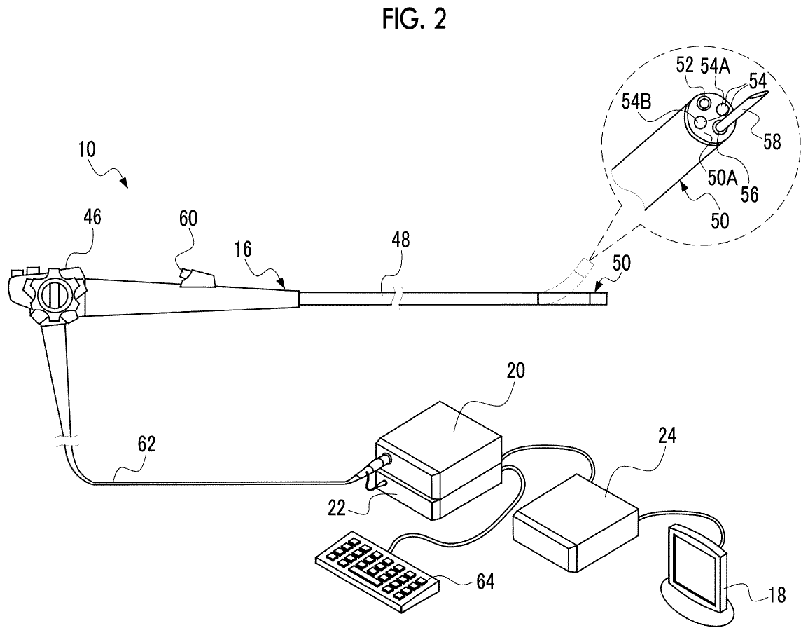

For example, as shown in FIG. 2, the endoscope 16 comprises an operating part 46 and an insertion part 48. The insertion part 48 is partially bent by the operation of the operating part 46. The insertion part 48 is inserted into the large intestine 28 while being bent along the shape of the large intestine 28 (see FIG. 1) in accordance with the operation of the operating part 46 performed by the doctor 12 (see FIG. 1).

A camera 52, an illumination device 54, and a treatment tool opening 56 are provided at a distal end portion 50 of the insertion part 48. The camera 52 and the illumination device 54 are provided on a distal end surface 50A of the distal end portion 50. It should be noted that, here, the form example is described in which the camera 52 and the illumination device 54 are provided on the distal end surface 50A of the distal end portion 50, but this is merely an example, and the endoscope 16 may be configured as a side-view endoscope by providing the camera 52 and the illumination device 54 on a side surface of the distal end portion 50.

The camera 52 is inserted into the body cavity of the subject 26 and images the observation target region. In the present embodiment, the camera 52 is a device that acquires the endoscopic video image 39 as a medical image by imaging the inside of the body (for example, the inside of the large intestine 28) of the subject 26. Examples of the camera 52 include a CMOS camera. However, this is merely an example, and other types of cameras, such as CCD cameras, may be used.

The illumination device 54 includes illumination windows 54A and 54B. The illumination device 54 emits the light 30 (see FIG. 1) through the illumination windows 54A and 54B. Examples of the types of the light 30 emitted from the illumination device 54 include visible light (for example, white light) and invisible light (for example, near-infrared light). Furthermore, the illumination device 54 emits special light via the illumination windows 54A and 54B. Examples of the special light include light for BLI and/or light for LCI. The camera 52 images the inside of the large intestine 28 using an optical method in a state in which the illumination device 54 irradiates the inside of the large intestine 28 with the light 30.

The treatment tool opening 56 is an opening for allowing a treatment tool 58 to protrude from the distal end portion 50. Furthermore, the treatment tool opening 56 is also used as a suction port for suctioning blood, internal contaminants, and the like and a sending-out port for sending out fluid.

A treatment tool insertion port 60 is formed at the operating part 46, and the treatment tool 58 is inserted into the insertion part 48 through the treatment tool insertion port 60. The treatment tool 58 passes through the insertion part 48 to protrude from the treatment tool opening 56 to the outside. In the example shown in FIG. 2, an aspect is shown in which a biopsy needle protrudes through the treatment tool opening 56 as the treatment tool 58. Here, the biopsy needle has been described as an example of the treatment tool 58, but this is merely an example, and the treatment tool 58 may be grasping forceps, a papillotomy knife, a snare, a catheter, a guide wire, a cannula, and/or a biopsy needle with a guide sheath.

The endoscope 16 is connected to the light source device 20 and the control device 22 through a universal cord 62. The medical support device 24 and a reception device 64 are connected to the control device 22. Furthermore, the display device 18 is connected to the medical support device 24. That is, the control device 22 is connected to the display device 18 via the medical support device 24.

Here, since the medical support device 24 is used as an example of an external device for expanding the functions of the control device 22, the form example has been described in which the control device 22 and the display device 18 are indirectly connected to each other via the medical support device 24, but this is merely an example. For example, the display device 18 may be directly connected to the control device 22. In this case, for example, the functions of the medical support device 24 need only be provided in the control device 22, or the control device 22 need only have a function of directing a server (not shown) to execute the same processing as the processing (for example, the medical support processing which will be described later) executed by the medical support device 24, receiving a processing result obtained by the server, and using the processing result.

The reception device 64 receives an instruction from the doctor 12, and outputs the received instruction as an electric signal to the control device 22. Examples of the reception device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light to the illumination device 54. The illumination device 54 is provided with a built-in light guide, and the light supplied from the light source device 20 is emitted from the illumination windows 54A and 54B via the light guide. The control device 22 causes the camera 52 to perform imaging, acquires the endoscopic video image 39 (see FIG. 1) from the camera 52, and outputs the endoscopic video image 39 to a predetermined output destination (for example, the medical support device 24).

The medical support device 24 executes various types of image processing on the endoscopic video image 39 input from the control device 22 to support the medical treatment (here, for example, endoscopy). The medical support device 24 outputs the endoscopic video image 39 subjected to various types of image processing to a predetermined output destination (for example, the display device 18).

Here, the form example has been described in which the endoscopic video image 39 output from the control device 22 is output to the display device 18 via the medical support device 24, but this is merely an example. For example, an aspect may be adopted in which the control device 22 and the display device 18 are connected to each other, and the endoscopic video image 39 that has been subjected to the image processing by the medical support device 24 is displayed on the display device 18 via the control device 22.

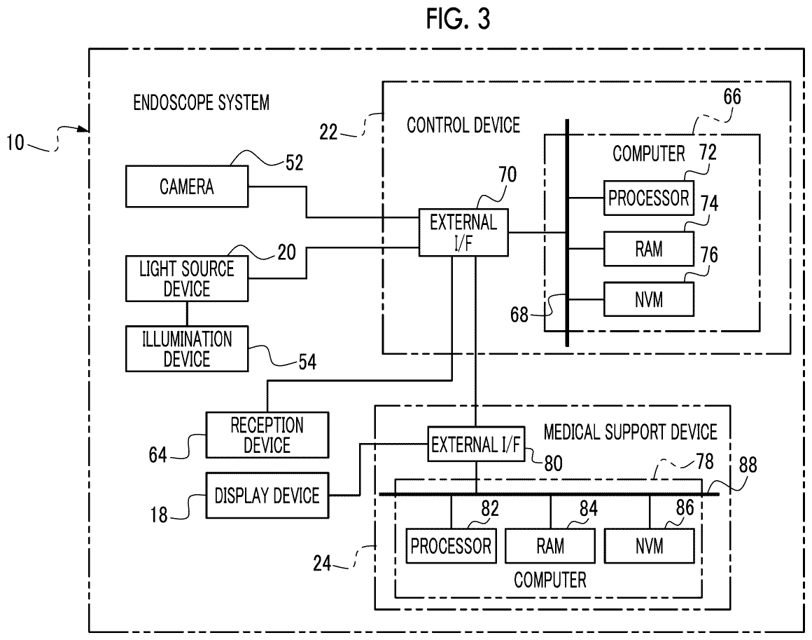

As shown in FIG. 3 as an example, the control device 22 comprises a computer 66, a bus 68, and an external I/F 70. The computer 66 comprises a processor 72, a RAM 74, and an NVM 76. The processor 72, the RAM 74, the NVM 76, and the external I/F 70 are connected to the bus 68.

The processor 72 includes, for example, at least one CPU and at least one GPU, and controls the entire control device 22. The GPU operates under the control of the CPU and is responsible for, for example, the execution of various types of processing of a graphic system and performing calculation using a neural network. The processor 72 may be one or more CPUs integrated with a GPU function, or may be one or more CPUs not integrated with the GPU function. Furthermore, the example shown in FIG. 3 shows an aspect in which one processor 72 is installed in the computer 66, but this is merely an example, and a plurality of processors 72 may be installed in the computer 66.

The RAM 74 is a memory that temporarily stores information, and is used as a working memory by the processor 72. The NVM 76 is a non-volatile storage device that stores various programs, various parameters, and the like. Examples of the NVM 76 include a flash memory (for example, an EEPROM and/or an SSD). The flash memory is merely an example, and may be another non-volatile storage device such as an HDD or a combination of two or more types of non-volatile storage devices.

The external I/F 70 transmits and receives various types of information between one or more devices (hereinafter, also referred to as “first external devices”) existing outside the control device 22 and the processor 72. Examples of the external I/F 70 include a USB interface.

The camera 52 is connected to the external I/F 70 as one of the first external devices, and the external I/F 70 transmits and receives various types of information between the camera 52 and the processor 72. The processor 72 controls the camera 52 through the external I/F 70. In addition, the processor 72 acquires, via the external I/F 70, the endoscopic video image 39 (see FIG. 1) obtained by imaging the inside of the large intestine 28 (see FIG. 1) via the camera 52.

The light source device 20 is connected to the external I/F 70 as one of the first external devices, and the external I/F 70 transmits and receives various types of information between the light source device 20 and the processor 72. The light source device 20 supplies the light to the illumination device 54 under the control of the processor 72. The illumination device 54 emits the light supplied from the light source device 20.

The reception device 64 is connected to the external I/F 70 as one of the first external devices, and the processor 72 acquires the instruction received by the reception device 64 via the external I/F 70 and executes the processing corresponding to the acquired instruction.

The medical support device 24 comprises a computer 78 and an external I/F 80. The computer 78 comprises a processor 82, a RAM 84, and an NVM 86. The processor 82, the RAM 84, the NVM 86, and the external I/F 80 are connected to a bus 88. In the present embodiment, the medical support device 24 is an example of a “medical support device” according to the technology of the present disclosure, the computer 78 is an example of a “computer” according to the technology of the present disclosure, and the processor 82 is an example of a “processor” according to the technology of the present disclosure.

Since a hardware configuration of the computer 78 (that is, the processor 82, the RAM 84, and the NVM 86) is essentially the same as a hardware configuration of the computer 66, the description of the hardware configuration of the computer 78 will be omitted here.

The external I/F 80 transmits and receives various types of information between one or more devices (hereinafter, also referred to as “second external devices”) existing outside the medical support device 24 and the processor 82. Examples of the external I/F 80 include a USB interface.

The control device 22 is connected to the external I/F 80 as one of the second external devices. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic video image 39 (see FIG. 1) from the processor 72 of the control device 22 via the external I/Fs 70 and 80, and executes various types of image processing on the acquired endoscopic video image 39.

The display device 18 is connected to the external I/F 80 as one of the second external devices. The processor 82 controls the display device 18 via the external I/F 80 such that various types of information (for example, the endoscopic video image 39 on which various types of image processing have been performed) are displayed on the display device 18.

Meanwhile, in the endoscopy, the doctor 12 determines whether or not the medical treatment is necessary for the lesion 42 shown in the endoscopic video image 39 while checking the endoscopic video image 39 via the display device 18, and performs the medical treatment for the lesion 42 in a case where the medical treatment is necessary. In a case of determining whether or not the medical treatment is required, the size of the lesion 42 is an important determination factor.

In recent years, with the development of machine learning, it has been possible to detect and discriminate the lesion 42 based on the endoscopic video image 39 by using an AI-based method. By applying this technology, it is possible to measure the size of the lesion 42 from the endoscopic video image 39. Measuring the size of the lesion 42 with high accuracy and presenting a measurement result to the doctor 12 are very useful for the doctor 12 to perform the medical treatment on the lesion.

However, in a case in which the size of the lesion 42 shown in the endoscopic video image 39 is displaced in the endoscopic video image 39 (that is, in a case in which the size of the lesion 42 in the endoscopic video image 39 is not stable) due to body movement and/or shaking of the camera 52, there is a concern that the size is erroneously measured and presented to the doctor 12.

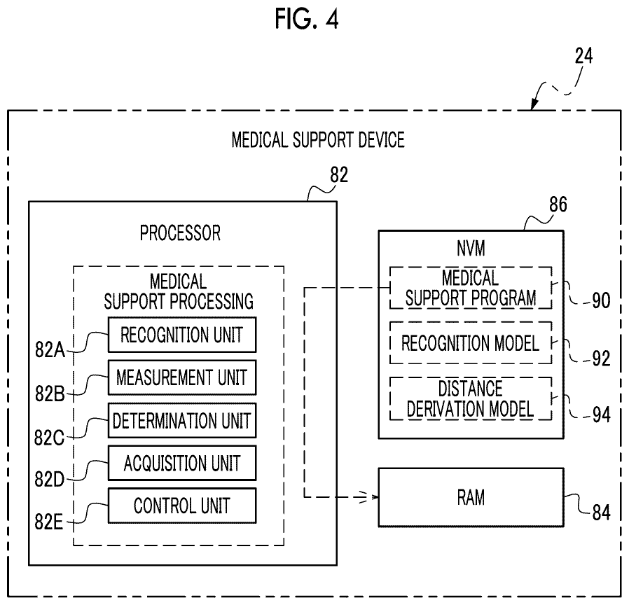

Therefore, in view of such circumstances, in the present embodiment, for example, as shown in FIG. 4, the processor 82 of the medical support device 24 executes the medical support processing.

The NVM 86 stores a medical support program 90. The medical support program 90 is an example of a “program” according to the technology of the present disclosure. The processor 82 reads out the medical support program 90 from the NVM 86 and executes the read out medical support program 90 on the RAM 84 to execute the medical support processing. The medical support processing is implemented by the processor 82 operating as a recognition unit 82A, a measurement unit 82B, a determination unit 82C, an acquisition unit 82D, and a control unit 82E in accordance with the medical support program 90 executed on the RAM 84.

The NVM 86 stores a recognition model 92 and a distance derivation model 94. As will be described in detail later, the recognition model 92 is used by the recognition unit 82A, and the distance derivation model 94 is used by the measurement unit 82B. The recognition model 92 in the present embodiment is an example of “AI” according to the technology of the present disclosure.

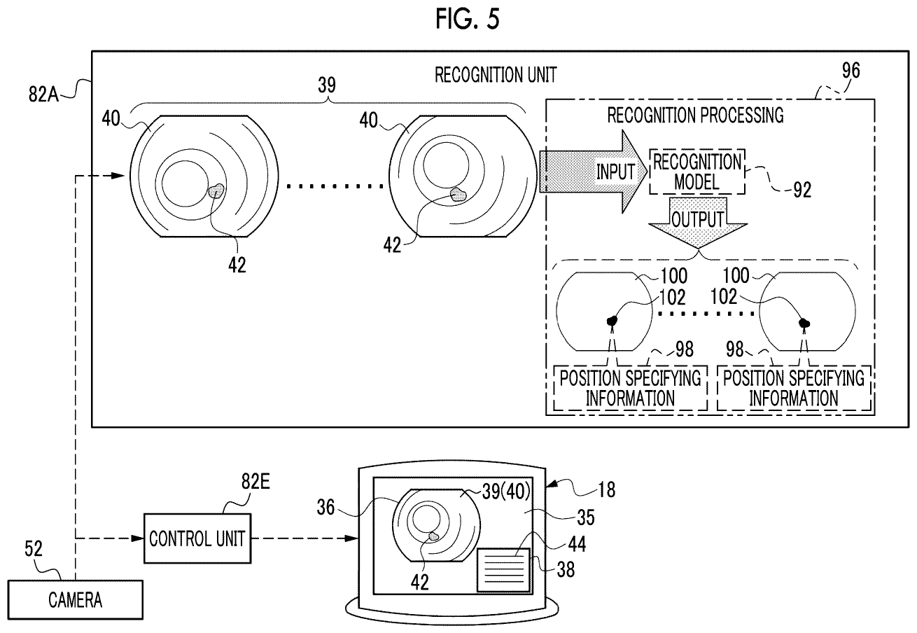

For example, as shown in FIG. 5, the recognition unit 82A and the control unit 82E acquire each of the plurality of frames 40, which are arranged in time series in the endoscopic video image 39 generated by being captured by the camera 52 in accordance with an imaging frame rate (for example, several tens of frames/second), from the camera 52 frame by frame in time series.

The control unit 82E displays the endoscopic video image 39 in the first display region 36 as a live view image. That is, each time the frame 40 is acquired from the camera 52, the control unit 82E displays the acquired frames 40 in the first display region 36 in order at a display frame rate (for example, several tens of frames/second).

The recognition unit 82A recognizes the lesion 42 in the endoscopic video image 39, by using the endoscopic video image 39 acquired from the camera 52. That is, the recognition unit 82A sequentially performs recognition processing 96 on each of the plurality of frames 40 arranged in time series in the endoscopic video image 39 acquired from the camera 52, to recognize the lesion 42 shown in the frame 40. For example, the recognition unit 82A recognizes the geometric characteristics (for example, the position and the shape) of the lesion 42, the type of the lesion 42, and the subtype of the lesion 42 (for example, a pedunculated type, a sub-pedunculated type, a sessile type, a surface protrusion type, a surface flat type, and a surface concave type).

The recognition processing 96 is performed on the acquired frame 40 each time the recognition unit 82A acquires the frame 40. The recognition processing 96 is processing of recognizing the lesion 42 with a method using AI. In the present embodiment, for example, object recognition processing (for example, semantic segmentation, instance segmentation, and/or panoptic segmentation) by an AI-based segmentation method is used as the recognition processing 96.

Here, as the recognition processing 96, processing using the recognition model 92 is performed. The recognition model 92 is a trained model for object recognition using the AI-based segmentation method. Examples of the trained model for object recognition using the AI-based segmentation method include a model for semantic segmentation. Examples of the model for semantic segmentation include a model with an encoder-decoder architecture. Examples of the model with the encoder-decoder architecture include a U-Net and an HRNet.

The recognition model 92 has been optimized by training a neural network through machine learning using first training data. The first training data is a dataset including a plurality of data (that is, data for a plurality of frames) in which first example data is associated with first ground truth data.

The first example data is an image corresponding to the frame 40. The first ground truth data is ground truth data (that is, an annotation) for the first example data. Here, an annotation for specifying the geometric characteristics, the type, and the subtype of the lesion shown in the image used as the first example data is used as an example of the first ground truth data.

The recognition unit 82A acquires the frame 40 from the camera 52, and inputs the acquired frame 40 to the recognition model 92. Therefore, each time the frame 40 is input, the recognition model 92 specifies the geometric characteristics of the lesion 42 shown in the input frame 40, and outputs information that can specify the geometric characteristics. In the example shown in FIG. 5, position specifying information 98 that can specify the position of the lesion 42 in the frame 40 is shown as an example of the information that can specify the geometric characteristics. In addition, the recognition unit 82A acquires information indicating the type and the subtype of the lesion 42 shown in the frame 40 input to the recognition model 92 from the recognition model 92.

The recognition unit 82A acquires a probability map 100 related to the frame 40 input to the recognition model 92 from the recognition model 92 each time the frame 40 is input to the recognition model 92. The probability map 100 is a map in which the distribution of the position of the lesion 42 in the frame 40 is represented by a probability that is an example of an indicator indicating a likelihood. It should be noted that, in general, the probability map 100 is also referred to as a reliability map, a confidence level map, or the like.

The probability map 100 includes a segmentation image 102 that defines the lesion 42 recognized by the recognition unit 82A. The segmentation image 102 is an image region that specifies the position of the lesion 42, which is recognized by performing the recognition processing 96 on the frame 40, in the frame 40 (that is, an image displayed in a display aspect in which the position in the frame 40 at which the lesion 42 is most likely to exist can be specified). The position specifying information 98 is associated with the segmentation image 102 by the recognition unit 82A. Examples of the position specifying information 98 in this case include coordinates for specifying a position of the segmentation image 102 in the frame 40. The segmentation image 102 in the present embodiment is an example of a “closed region” and a “segmentation image” according to the technology of the present disclosure.

Although not shown in FIG. 5, the probability map 100 may be displayed on the screen 35 (for example, the second display region 38) by the control unit 82E. In such a case, the probability map 100 displayed on the screen 35 is updated in accordance with the display frame rate applied to the first display region 36. That is, the display of the probability map 100 (that is, the display of the segmentation image 102) in the second display region 38 is updated in synchronization with the display time of the endoscopic video image 39 displayed in the first display region 36. With such a configuration, the doctor 12 can ascertain an approximate position of the lesion 42 in the endoscopic video image 39 displayed in the first display region 36 by observing the endoscopic video image 39 displayed in the first display region 36 and referring to the probability map 100 displayed in the second display region 38.

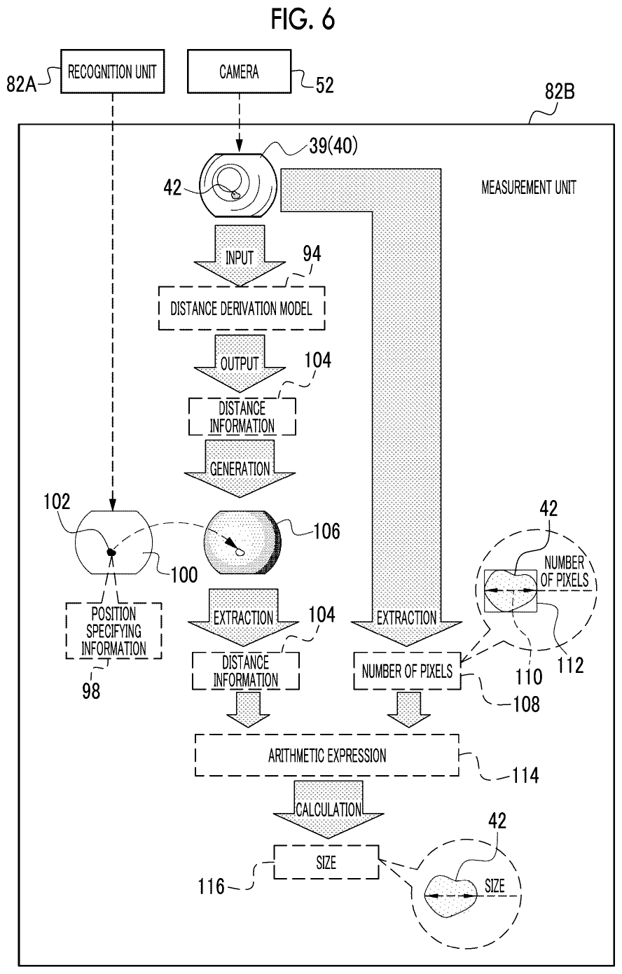

As shown in FIG. 6 as an example, the measurement unit 82B measures sizes 116 of the lesion 42 in time series based on each of the plurality of frames 40 included in the endoscopic video image 39 acquired from the camera 52. The size 116 of the lesion 42 means a size of the lesion 42 in the real space. Hereinafter, for convenience of description, the size of the lesion 42 in the real space will also be referred to as a “real size”.

In order to implement the measurement of the size 116 of the lesion 42, the measurement unit 82B acquires distance information 104 of the lesion 42 based on the frame 40 acquired from the camera 52. The distance information 104 is information indicating a distance from the camera 52 (that is, the observation position) to the intestinal wall 32 (see FIG. 1) including the lesion 42. It should be noted that, here, the distance from the camera 52 to the intestinal wall 32 including the lesion 42 has been described as an example, but this is merely an example, and a numerical value indicating a depth from the camera 52 to the intestinal wall 32 including the lesion 42 (for example, a plurality of numerical values that define the depth in stages (for example, numerical values from several stages to several tens of stages)) may be used instead of the distance.

The distance information 104 is acquired for each pixel constituting the frame 40. It should be noted that the distance information 104 may be acquired for each block (for example, a pixel group composed of several pixels to several hundred pixels) larger than a pixel in the frame 40.

The acquisition of the distance information 104 by the measurement unit 82B is implemented, for example, by deriving the distance information 104 by using an AI-based method. In the present embodiment, the distance derivation model 94 is used to derive the distance information 104.

The distance derivation model 94 is optimized by training a neural network through machine learning using second training data. The second training data is a dataset including a plurality of data (that is, data for a plurality of frames) in which second example data is associated with second ground truth data.

The second example data is an image corresponding to the frame 40. The second ground truth data is ground truth data (that is, an annotation) for the second example data. Here, an annotation for specifying the distance corresponding to each pixel included in the image used as the second example data is used as an example of the second ground truth data.

The measurement unit 82B acquires the frame 40 from the camera 52, and inputs the acquired frame 40 to the distance derivation model 94. As a result, the distance derivation model 94 outputs the distance information 104 in units of pixels of the input frame 40. That is, in the measurement unit 82B, information indicating the distance from the position of the camera 52 (for example, the position of the image sensor or the objective lens installed in the camera 52) to the intestinal wall 32 shown in the frame 40 is output from the distance derivation model 94 as the distance information 104 in units of pixels of the frame 40.

The measurement unit 82B generates a distance image 106 based on the distance information 104 output from the distance derivation model 94. The distance image 106 is an image in which the distance information 104 is distributed in units of pixels included in the endoscopic video image 39.

The measurement unit 82B acquires the position specifying information 98 added to a segmentation image 102 in the probability map 100 obtained by the recognition unit 82A. The measurement unit 82B extracts the distance information 104 corresponding to the position specified from the position specifying information 98 from the distance image 106, with reference to the position specifying information 98. Examples of the distance information 104 extracted from the distance image 106 include the distance information 104 corresponding to a position (for example, a centroid) of the lesion 42 or a statistic value (for example, a median value, an average value, or a mode value) of the distance information 104 for a plurality of pixels (for example, all pixels) included in the lesion 42.

The measurement unit 82B extracts the number of pixels 108 from the frame 40. The number of pixels 108 is the number of pixels on a line segment 110 that crosses an image region (that is, an image region showing the lesion 42) at the position specified from the position specifying information 98 in the entire image region of the frame 40 input to the distance derivation model 94. Examples of the line segment 110 include a longest line segment parallel to a long side of a circumscribing rectangular frame 112 in the image region showing the lesion 42. It should be noted that the line segment 110 is merely an example, and a longest line segment parallel to a short side of the circumscribing rectangular frame 112 in the image region indicating the lesion 42 may be applied instead of the line segment 110.

The measurement unit 82B calculates the size 116 of the lesion 42 based on the distance information 104 extracted from the distance image 106 and the number of pixels 108 extracted from the frame 40. An arithmetic expression 114 is used to calculate the size 116. The measurement unit 82B inputs the distance information 104 extracted from the distance image 106 and the number of pixels 108 extracted from the frame 40 to the arithmetic expression 114. The arithmetic expression 114 is an arithmetic expression in which the distance information 104 and the number of pixels 108 are independent variables and the size 116 is a dependent variable. The arithmetic expression 114 outputs the size 116 corresponding to the input distance information 104 and the input number of pixels 108.

In addition, here, the length of the lesion 42 in the real space has been described as the size 116, but the technology of the present disclosure is not limited to this, and the size 116 may be a surface area or a volume of the lesion 42 in the real space. In this case, as the arithmetic expression 114, for example, an arithmetic expression is used in which the number of pixels of the entire image region indicating the lesion 42 and the distance information 104 are independent variables and the surface area or volume of the lesion 42 in the real space is a dependent variable.

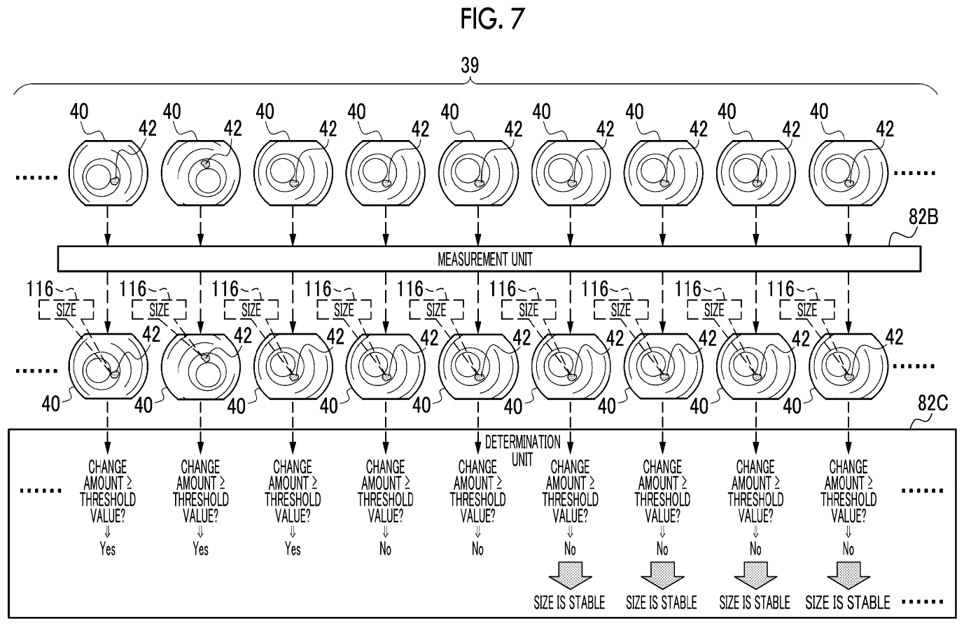

As shown in FIG. 7 as an example, the determination unit 82C acquires the size 116 from the measurement unit 82B each time the size 116 is measured by the measurement unit 82B. Then, the determination unit 82C determines whether or not the sizes 116 in the time series are stable based on the measurement result of the size 116 obtained by the measurement unit 82B (that is, the size 116 acquired from the measurement unit 82B).

The determination of whether or not the sizes 116 in the time series are stable is performed by calculating a size change amount. The size change amount refers to a change amount of the size 116 of the lesion 42 between the frames 40 adjacent in time series. The determination unit 82C calculates the size change amount from two sizes 116 measured from the frames 40 adjacent in time series, and determines whether or not the calculated size change amount is equal to or greater than a threshold value. The threshold value may be a fixed value or a variable value that is changed in accordance with the instruction and/or the imaging condition received by the reception device 64 by the user or the like.

In a case in which the size change amount is not less than the threshold value for three consecutive frames during a period in which three frames 40 are consecutive in time series, the determination unit 82C determines that the sizes 116 in the time series are not stable. In addition, in a case in which the size change amount is less than the threshold value for the three consecutive frames during the period in which the three frames 40 are consecutive in time series, the determination unit 82C determines that the sizes 116 in the time series are stable. In the present embodiment, a period in which the three frames 40 are consecutive in time series is an example of a “second period” according to the technology of the present disclosure.

Here, although the form example has been described in which the determination of whether or not the size change amount is less than the threshold value is performed for the three consecutive frames, this is merely an example, and the determination of whether or not the size change amount is less than the threshold value may be performed for two consecutive frames, or the determination of whether or not the size change amount is less than the threshold value may be performed for four or more consecutive frames. In addition, the determination of whether or not the size change amount is less than the threshold value may be performed in a single frame. In addition, the determination of whether or not the size change amount is less than the threshold value may be performed for a fixed number of consecutive frames or a fixed number of single frames, or the determination of whether or not the size change amount is less than the threshold value may be performed for the number of consecutive frames or the number of single frames, which is changed in accordance with a given instruction and/or various conditions.

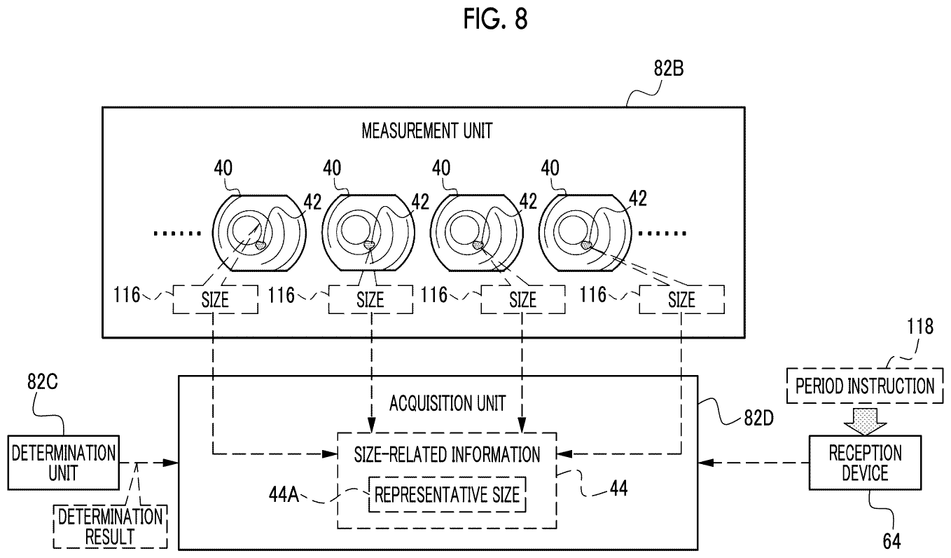

As shown in FIG. 8 as an example, the reception device 64 receives a period instruction 118 that is an instruction to determine the period. Examples of the period determined by the period instruction 118 include a period determined by the doctor 12 (for example, a period designated within a period in which the medical support processing is performed). Examples of the period determined by the doctor 12 include several seconds to several tens of seconds.

The acquisition unit 82D acquires each size 116 of the lesion 42 shown in each of the plurality of frames 40 from the measurement unit 82B based on the determination result obtained by the determination unit 82C (that is, a result of determining whether or not the sizes 116 in the time series are stable) within the period determined by the period instruction 118 received by the reception device 64.

For example, in a case in which the determination unit 82C determines that the sizes 116 in the time series are stable within the period determined by the period instruction 118 received by the reception device 64, the acquisition unit 82D acquires the sizes 116 of the plurality of frames 40 from the measurement unit 82B. More specifically, the acquisition unit 82D acquires the sizes 116 of the lesion 42 (hereinafter, also referred to as a “plurality of sizes 116”) shown in a plurality of consecutive frames 40 for which it is determined by the determination unit 82C that the sizes 116 in the time series are stable within the period determined by the period instruction 118 received by the reception device 64, from the measurement unit 82B using an FIFO method. Examples of the plurality of sizes 116 acquired by the acquisition unit 82D using the FIFO method include the sizes 116 of several frames to several hundred frames.

The acquisition unit 82D acquires the size-related information 44 based on the plurality of sizes 116 acquired from the measurement unit 82B. The size-related information 44 is information corresponding to the sizes 116 in the time series. The acquisition of the size-related information 44 is implemented by calculating the size-related information 44 via the acquisition unit 82D based on the plurality of sizes 116 acquired from the measurement unit 82B.

It should be noted that, here, although the form example has been described in which the size-related information 44 is calculated by the acquisition unit 82D of the medical support device 24, this is merely an example, and the size-related information 44 calculated by a device other than the medical support device 24 (for example, the control device 22 or a device (for example, a server, a personal computer, and/or a tablet terminal) that is communicably connected to the endoscope system 10) may be acquired by the acquisition unit 82D.

A representative size 44A is used as the size-related information 44. The representative size 44A is a real size that is representative of the plurality of sizes 116 acquired by the acquisition unit 82D from the measurement unit 82B. Examples of the representative size 44A include an average value of the sizes 116 within the period determined by the period instruction 118, a minimum value of the size 116 within the period determined by the period instruction 118, a maximum value of the size 116 within the period determined by the period instruction 118, and the size 116 at a stable moment. The size 116 at the stable moment refers to, for example, a latest size 116 in a case in which it is determined by the determination unit 82C that the sizes 116 are stable (that is, the latest size 116 used to calculate the size change amount compared with the threshold value in a case in which it is determined by the determination unit 82C that the sizes 116 are stable).

It should be noted that, here, as an example of the representative size 44A, the average value of the sizes 116 within the period determined by the period instruction 118, the minimum value within the period determined by the period instruction 118, the maximum value within the period determined by the period instruction 118, and the size 116 at the stable moment have been described, but this is merely an example. For example, the representative size 44A may be the average value of the sizes 116 within the period determined by the period instruction 118, the minimum value within the period determined by the period instruction 118, the maximum value within the period determined by the period instruction 118, the size 116 at the stable moment, the frequency of the sizes 116 within the period determined by the period instruction 118, a median value of the sizes 116 within the period determined by the period instruction 118, and/or a variance value of the sizes 116 within the period determined by the period instruction 118. Further, the representative size 44A may be one or more statistical values other than the average value, the minimum value, the maximum value, the frequency, the median value, and the variance value.

The size-related information 44 in the present embodiment is an example of “size-related information” according to the technology of the present disclosure. In addition, the period determined by the period instruction 118 in the present embodiment is an example of a “first period” according to the technology of the present disclosure. In addition, the representative size 44A in the present embodiment is an example of a “representative value” according to the technology of the present disclosure.

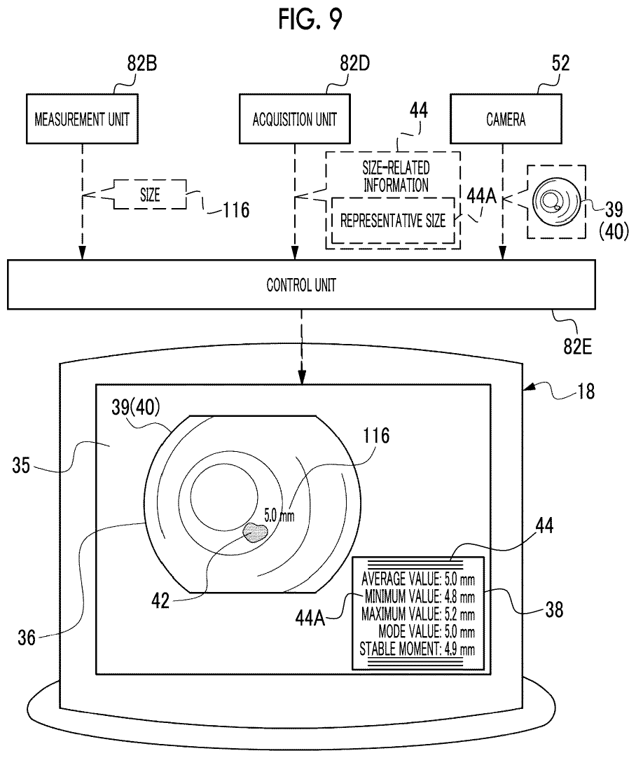

As shown in FIG. 9 as an example, the control unit 82E acquires the size 116 from the measurement unit 82B. Further, the control unit 82E acquires the size-related information 44 from the acquisition unit 82D.

The control unit 82E displays the endoscopic video image 39 in the first display region 36, and displays the size 116 acquired from the measurement unit 82B in the endoscopic video image 39. For example, the size 116 is displayed superimposed on the endoscopic video image 39. It should be noted that the superimposed display is merely an example, and may be an embedded display. In addition, in a case in which the size 116 is displayed superimposed on the endoscopic video image 39, the size 116 may be displayed superimposed on the endoscopic video image 39 using an alpha blending method.

In addition, in a case in which the determination unit 82C determines that the sizes 116 in the time series are stable, the control unit 82E displays the size-related information 44 acquired from the acquisition unit 82D in the second display region 38. The representative size 44A is used in the size-related information 44, and thus the representative size 44A is displayed in the second display region 38.

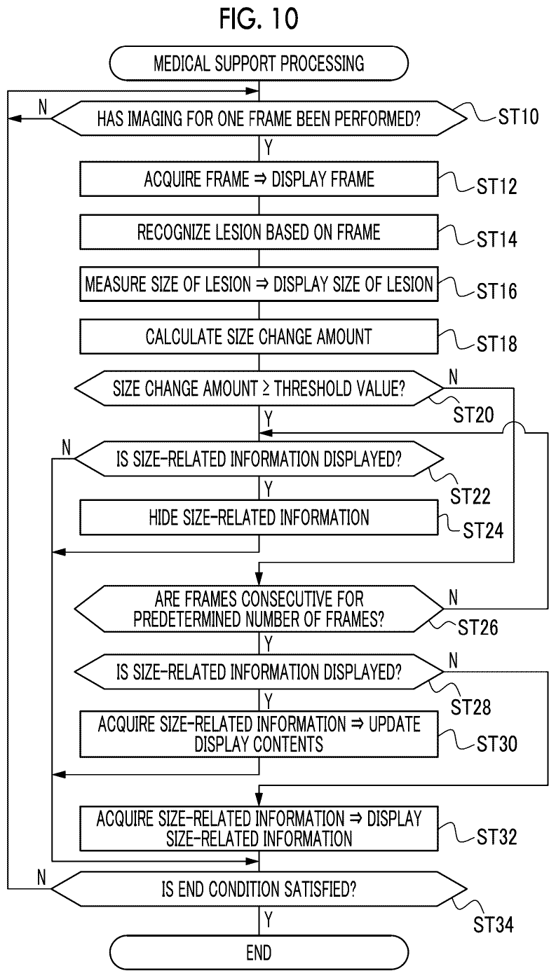

Hereinafter, the operation of the portion of the endoscope system 10 according to the technology of the present disclosure will be described with reference to FIG. 10. The flow of the medical support processing shown in FIG. 10 is an example of a “medical support method” according to the technology of the present disclosure.

In the medical support processing shown in FIG. 10, first, in step ST10, the recognition unit 82A determines whether or not imaging for one frame has been performed by the camera 52 in the large intestine 28. In step ST10, in a case in which the imaging for one frame has not been performed by the camera 52 in the large intestine 28, a negative determination is made, and the determination in step ST10 is performed again. In step ST10, in a case in which the imaging for one frame has been performed by the camera 52 in the large intestine 28, an affirmative determination is made, and the medical support processing proceeds to step ST12.

In step ST12, the recognition unit 82A and the control unit 82E acquire the frame 40 obtained by imaging the large intestine 28 with the camera 52. The control unit 82E displays the frame 40 in the first display region 36 (see FIGS. 5 and 9). It should be noted that, here, for convenience of description, the description will be made on the premise that the lesion 42 is shown in the endoscopic video image 39. After the processing of step ST12 is executed, the medical support processing proceeds to step ST14.

In step ST14, the recognition unit 82A recognizes the lesion 42 in the frame 40 by performing the recognition processing 96 using the frame 40 acquired in step ST12 (see FIG. 5). After the processing of step ST14 is executed, the medical support processing proceeds to step ST16.

In step ST16, the measurement unit 82B measures the size 116 of the lesion 42 shown in the frame 40 acquired in step ST12 based on the recognition result in step ST14 (see FIG. 6). The control unit 82E displays the size 116 measured by the measurement unit 82B in the frame 40 displayed in the first display region 36 (see FIG. 9). After the processing of step ST16 is executed, the medical support processing proceeds to step ST18.

In step ST18, the determination unit 82C calculates the size change amount by using the size 116 measured in step ST14 (see FIG. 7). After the processing in step ST18 is executed, the medical support processing proceeds to step ST20.

In step ST20, the determination unit 82C determines whether or not the size change amount calculated in step ST18 is equal to or greater than the threshold value (see FIG. 7). In step ST20, in a case in which the size change amount calculated in step ST18 is equal to or greater than the threshold value, an affirmative determination is made, and the medical support processing proceeds to step ST22. In step ST20, in a case in which the size change amount calculated in step ST18 is less than the threshold value, a negative determination is made, and the medical support processing proceeds to step ST26.

In step ST22, the control unit 82E determines whether or not the size-related information 44 is displayed in the second display region 38. In step ST22, in a case in which the size-related information 44 is displayed in the second display region 38, an affirmative determination is made, and the medical support processing proceeds to step ST24. In step ST22, in a case in which the size-related information 44 is not displayed in the second display region 38, a negative determination is made, and the medical support processing proceeds to step ST34.

In step ST24, the control unit 82E hides the size-related information 44 in the second display region 38. After the processing in step ST24 is executed, the medical support processing proceeds to step ST34.

In step ST26, the acquisition unit 82D determines whether or not the frames for which it is determined that the size change amount is equal to or greater than the threshold value are consecutive for the predetermined number of frames (for example, the number of frames designated within a range of several frames to several hundred frames). In step ST26, in a case in which the number of frames for which it is determined that the size change amount is equal to or greater than the threshold value is equal to or greater than the predetermined number of frames, an affirmative determination is made, and the medical support processing proceeds to step ST28. In step ST26, in a case in which the number of frames for which it is determined that the size change amount is equal to or greater than the threshold value is less than the predetermined number of frames, a negative determination is made, and the medical support processing proceeds to step ST22.

In step ST28, the control unit 82E determines whether or not the size-related information 44 is displayed in the second display region 38. In step ST28, in a case in which the size-related information 44 is displayed in the second display region 38, an affirmative determination is made, and the medical support processing proceeds to step ST30. In step ST28, in a case in which the size-related information 44 is not displayed in the second display region 38, a negative determination is made, and the medical support processing proceeds to step ST32.

In step ST30, the acquisition unit 82D acquires, from the measurement unit 82B, the sizes 116 for the predetermined number of frames for which it is determined that the size change amount is equal to or greater than the threshold value, and acquires the size-related information 44 based on the sizes 116 for the predetermined number of frames for which it is determined that the size change amount is equal to or greater than the threshold value (see FIG. 8). The control unit 82E updates the display contents of the second display region 38 by replacing the size-related information 44 displayed in the second display region 38 with latest size-related information 44 acquired by the acquisition unit 82D. After the processing in step ST30 is executed, the medical support processing proceeds to step ST34.

In step ST32, the acquisition unit 82D acquires, from the measurement unit 82B, the sizes 116 for the predetermined number of frames for which it is determined that the size change amount is equal to or greater than the threshold value, and acquires the size-related information 44 based on the sizes 116 for the predetermined number of frames for which it is determined that the size change amount is equal to or greater than the threshold value (see FIG. 8). The control unit 82E displays the size-related information 44 acquired by the acquisition unit 82D in the second display region 38 (see FIG. 9). After the processing in step ST32 is executed, the medical support processing proceeds to step ST34.

In step ST34, the control unit 82E determines whether or not a medical support processing end condition is satisfied. Examples of the medical support processing end condition include a condition that an instruction to end the medical support processing is issued to the endoscope system 10 (for example, a condition that the reception device 64 receives the instruction to end the medical support processing).

In a case in which the medical support processing end condition is not satisfied in step ST34, a negative determination is made, and the medical support processing proceeds to step ST10. In a case in which the medical support processing end condition is satisfied in step ST34, an affirmative determination is made, and the medical support processing ends.

As described above, in the endoscope system 10 according to the present embodiment, the lesion 42 shown in the endoscopic video image 39 is recognized using the endoscopic video image 39 by the recognition unit 82A. In addition, the measurement unit 82B measures the sizes 116 of the lesion 42 in time series based on the endoscopic video image 39. In addition, the size-related information 44 that is information corresponding to the sizes 116 in the time series is acquired by the acquisition unit 82D. The size-related information 44, which is acquired by the acquisition unit 82D, is displayed in the second display region 38. The representative size 44A, which is a representative value of the sizes 116 in the time series, is used as the size-related information 44. Therefore, the doctor 12 can accurately ascertain the size 116 of the lesion 42 shown in the endoscopic video image 39.

In addition, in the endoscope system 10 according to the present embodiment, the value that is representative of the sizes 116 measured in time series based on the plurality of frames 40 included in the period determined by the period instruction 118 is acquired as the representative size 44A by the acquisition unit 82D. In the present embodiment, as the representative size 44A, the average value of the sizes 116 within the period determined by the period instruction 118, the minimum value within the period determined by the period instruction 118, the maximum value within the period determined by the period instruction 118, and the size 116 at the stable moment are used. The representative size 44A is displayed in the second display region 38. Therefore, the doctor 12 can accurately ascertain the sizes 116 of the lesion 42 shown in the plurality of frames 40 included in the period instruction 118.

In addition, in the endoscope system 10 according to the present embodiment, in a case in which the determination unit 82C determines that the sizes 116 in the time series are stable within the period determined by the period instruction 118 received by the reception device 64, the acquisition unit 82D acquires the size-related information 44. Therefore, at a timing at which the sizes 116 in the time series of the lesion 42 shown in the endoscopic video image 39 are stable, the doctor 12 can accurately ascertain the size 116 of the lesion 42 shown in the endoscopic video image 39.

In addition, in the endoscope system 10 according to the present embodiment, in a case in which the size change amount is less than the threshold value for the three consecutive frames during the period in which the three frames 40 are consecutive in time series, the determination unit 82C determines that the sizes 116 in the time series are stable. Therefore, the endoscope system 10 can accurately determine whether or not the sizes 116 in the time series of the lesion 42 shown in the endoscopic video image 39 are stable.

In addition, in the endoscope system 10 according to the present embodiment, the output of the size-related information 44 is implemented by being displayed in the second display region 38. Therefore, the doctor 12 can visually recognize the size 116 of the lesion 42 shown in the endoscopic video image 39.

In addition, in the endoscope system 10 according to the present embodiment, the size 116 measured by the measurement unit 82B is displayed superimposed on the endoscopic video image 39, and the size-related information 44 is displayed in the second display region 38 which is a separate display region from the endoscopic video image 39. Therefore, the doctor 12 can visually recognize the endoscopic video image 39 and the size-related information 44 with high visibility.

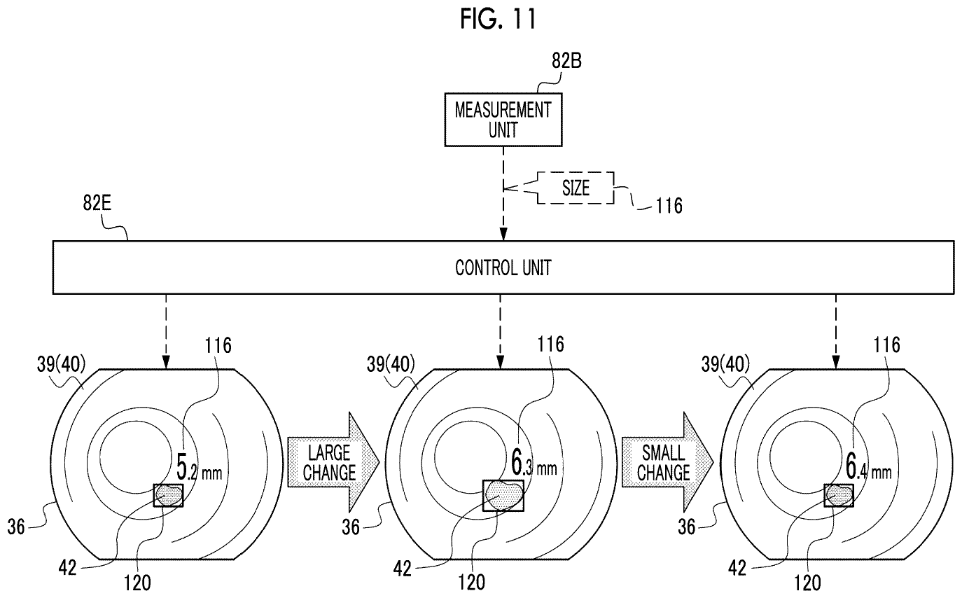

It should be noted that, in the above-described embodiment, although the form example has been described in which a font size of the real number is not changed in units of digits even in a case in which the size 116 displayed in the first display region 36 is the real number represented by the plurality of digits, the technology of the present disclosure is not limited to this. As shown in FIG. 11 as an example, in a case in which the size 116 displayed in the first display region 36 is a real number represented by a plurality of digits, the control unit 82E may change the font size of the real number in units of digits. In the example shown in FIG. 11, a font size of integer digits is larger than a font size of decimal digits. Therefore, in a case in which a value of the integer digit increases as the size 116 increases, the doctor 12 can visually recognize that the change in the size 116 is relatively large (that is, there is a high probability that the size 116 is not stable). In addition, in a case in which the value of the integer digit does not change and the value of the decimal digit increases as the size 116 increases, the doctor 12 can visually recognize that the change in the size 116 is relatively small (that is, the sizes 116 are likely to be stable).

The form example in which the font size is changed in units of digits is merely an example, and the font size, a font color, and/or font brightness may be changed in units of digits. In this case as well, the integer digits are made more prominent than the decimal digits.

In addition, in the example shown in FIG. 11, a circumscribing rectangular frame 120 for the image region of the lesion 42 corresponding to the displayed size 116 is displayed in the endoscopic video image 39. The circumscribing rectangular frame 120 may be generated based on the segmentation image 102 (see FIG. 5), or may be generated based on a bounding box obtained by performing object recognition processing using a bounding box method.

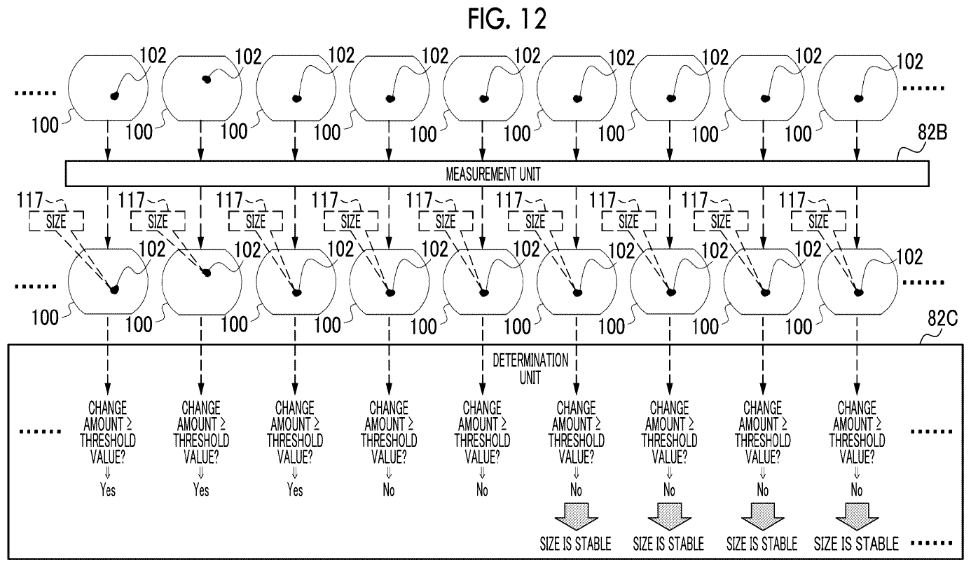

In the above-described embodiment, the form example has been described in which the size 116 of the lesion 42 is measured based on the frame 40, and it is determined whether or not the size change amount based on the size 116 is equal to or greater than the threshold value, but the technology of the present disclosure is not limited to this. For example, as shown in FIG. 12, a size 117 of the segmentation image 102 may be measured by the measurement unit 82B, and the determination unit 82C may determine whether or not the change amount of the size 117 is equal to or greater than the threshold value. The change amount of the size 117 may be calculated in the same manner as the calculation of the size change amount shown in the above-described embodiment. As described above, since the determination unit 82C determines whether or not the size 117 of the segmentation image 102 is equal to or greater than the threshold value, it is possible to easily specify whether or not the real size of the lesion 42 shown in the frame 40 is stable.

It should be noted that, here, although the form example has been described in which the size 117 of the segmentation image 102 is measured, this is merely an example, and in a case in which the recognition processing 96 is performed by an AI-based bounding box method, a change amount of a size of the bounding box, which is a closed region, may be calculated and compared with the threshold value. In addition, both the change amount of the size 117 of the segmentation image 102 and the change amount of the size of the bounding box may be calculated and compared with the threshold value. In this case as well, the same effects can be expected.

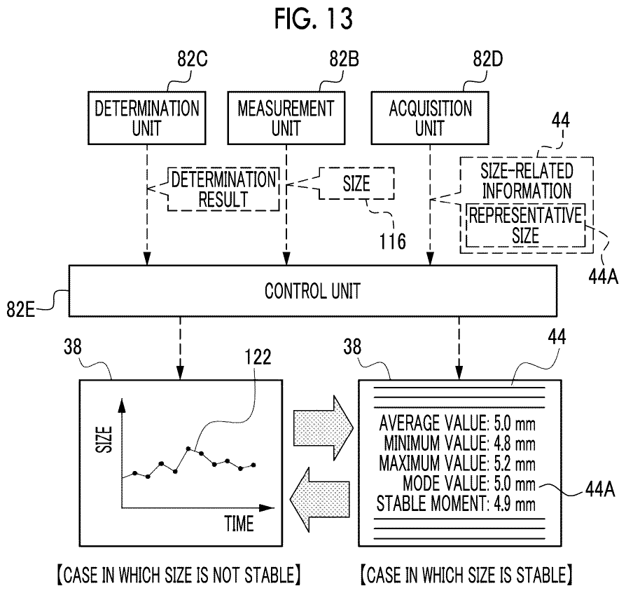

In the above-described embodiment, although the form example has been described in which the size-related information 44 is displayed in the second display region 38, the technology of the present disclosure is not limited to this. For example, as shown in FIG. 13, the control unit 82E may selectively display temporal change information 122 and the size-related information 44 in the second display region 38. The temporal change information 122 refers to information that can specify a temporal change of the sizes 116 in the time series. FIG. 13 shows a line graph in which the sizes 116 in the time series are plotted as an example of the temporal change information 122.

The control unit 82E selectively displays the temporal change information 122 and the size-related information 44 in the second display region 38 based on the determination result obtained by the determination unit 82C. For example, in a case in which the determination unit 82C determines that the sizes 116 in the time series are not stable, the control unit 82E displays the temporal change information 122 in the second display region 38. In addition, in a case in which the determination unit 82C determines that the sizes 116 in the time series are stable, the size-related information 44 is displayed in the second display region 38. That is, the display contents of the second display region 38 are switched from one of the temporal change information 122 or the size-related information 44 to the other in accordance with the determination result obtained by the determination unit 82C. As a result, the doctor 12 can easily ascertain whether or not the sizes 116 in the time series are stable by checking whether the temporal change information 122 or the size-related information 44 is displayed in the second display region 38.

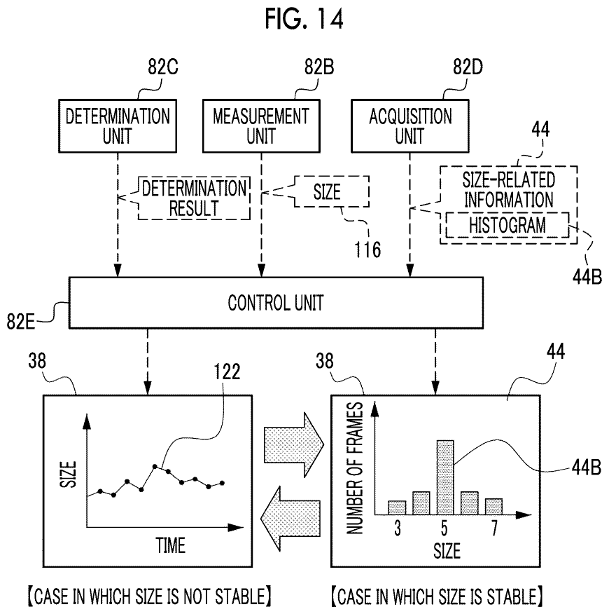

In the example shown in FIG. 13, the representative size 44A is used as the size-related information 44, but the technology of the present disclosure is not limited to this. As shown in FIG. 14 as an example, a histogram 44B may be used as the size-related information 44. The histogram 44B refers to, for example, a histogram of the frequency of the sizes 116 within the period determined by the period instruction 118. As described above, in the example shown in FIG. 14, since the histogram 44B is used as the size-related information 44, the doctor 12 can easily ascertain whether or not the sizes 116 in the time series of the lesion 42 in the plurality of frames 40 arranged in time series and included in the period determined by the period instruction 118 are stable by checking the histogram 44B displayed in the second display region 38.

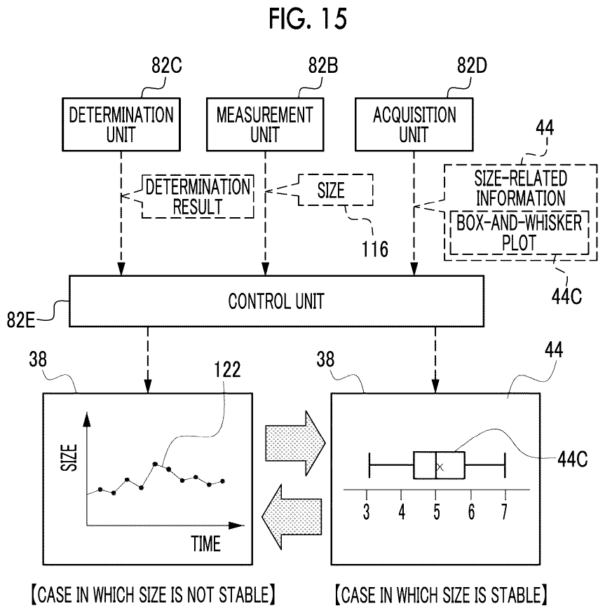

The histogram 44B shown in FIG. 14 is merely an example, and the size-related information 44 may use fluctuation range information indicating a fluctuation range from the maximum value within the period determined by the period instruction 118 to the minimum value within the period determined by the period instruction 118. Examples of the fluctuation range information include a box-and-whisker plot 44C shown in FIG. 15. The box-and-whisker plot 44C is a diagram in which a fluctuation range or the like from the maximum value within the period determined by the period instruction 118 to the minimum value within the period determined by the period instruction 118 is expressed. As described above, in the example shown in FIG. 15, since the box-and-whisker plot 44C is used as the size-related information 44, the doctor 12 can easily ascertain whether or not the sizes 116 in the time series of the lesion 42 in the plurality of frames 40 arranged in time series and included in the period determined by the period instruction 118 are stable by checking the box-and-whisker plot 44C displayed in the second display region 38.

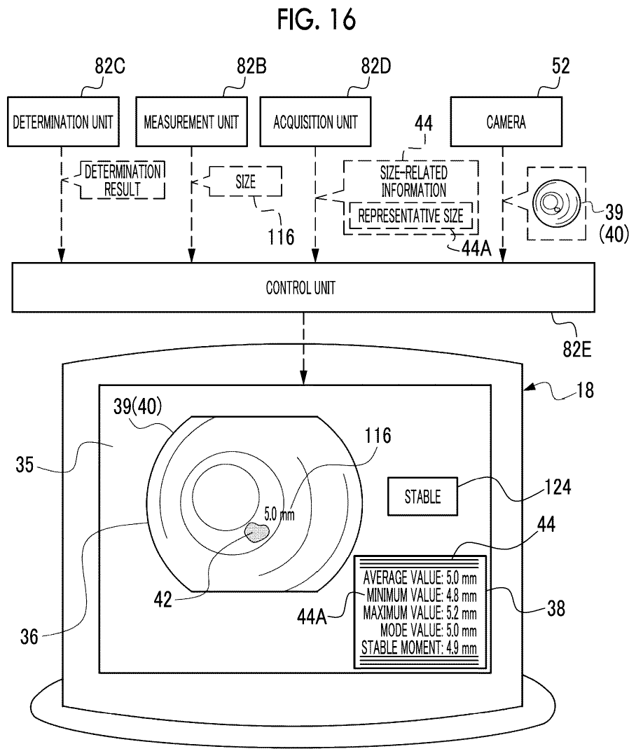

In the above-described embodiment, the form example has been described in which the doctor 12 visually ascertains whether or not the sizes 116 in the time series are stable from the size-related information 44 displayed in the second display region 38, but the technology of the present disclosure is not limited to this. For example, as shown in FIG. 16, the control unit 82E may output determination result information 124 indicating whether or not the sizes 116 in the time series are stable, with reference to the determination result obtained by the determination unit 82C. In the example shown in FIG. 16, as an example of the determination result information 124, information (here, for example, text information) indicating that the sizes 116 in the time series are stable is displayed on the screen 35. In addition, in a case in which the sizes 116 in the time series are not stable, information indicating that the sizes 116 in the time series are not stable is displayed on the screen 35 as the determination result information 124. As described above, the determination result information 124 is displayed on the screen 35, so that the doctor 12 can easily ascertain whether or not the sizes 116 in the time series are stable.

It should be noted that, here, although the form example has been described in which the information indicating that the sizes 116 in the time series are not stable is displayed on the screen 35 in a case in which the sizes 116 in the time series are not stable, the determination result information 124 may not be displayed on the screen 35 in a case in which the sizes 116 in the time series are not stable. In this case, the doctor 12 can easily recognize that the sizes 116 in the time series are not stable by checking that the determination result information 124 is not displayed on the screen 35.

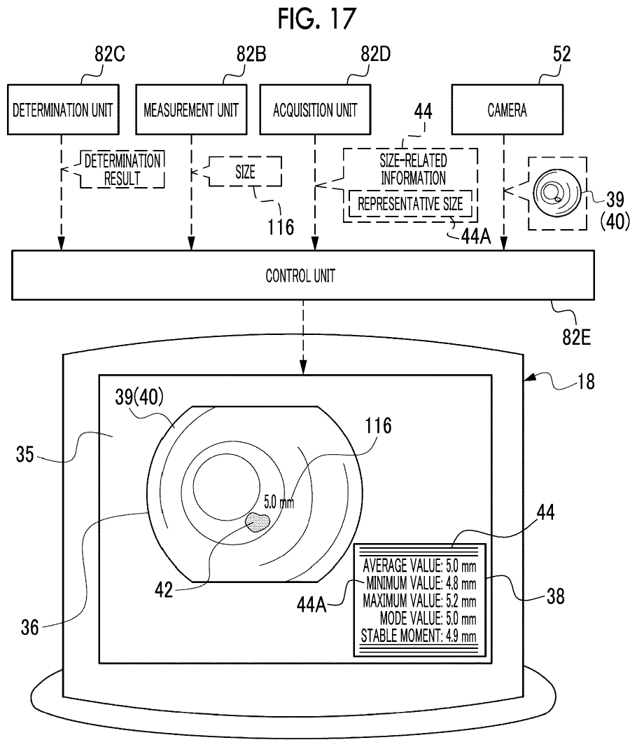

In the example shown in FIG. 16, the form example has been described in which the determination result information 124 is displayed on the screen 35 so that the doctor 12 can ascertain whether or not the sizes 116 in the time series are stable, but the technology of the present disclosure is not limited to this. For example, the control unit 82E may change the display aspect of the size-related information 44 in the second display region 38 in accordance with the determination result (that is, whether or not the sizes 116 in the time series are stable) obtained by the determination unit 82C. For example, in a case in which the determination unit 82C determines that the sizes 116 in the time series are stable, the representative size 44A is displayed in bold characters as shown in FIG. 16, and in a case in which the determination unit 82C determines that the sizes 116 in the time series are not stable, the representative size 44A is displayed in thin characters as shown in FIG. 17. This is merely an example, and the display aspect of the size-related information 44 may be changed by the control unit 82E such that the size-related information 44 displayed in the second display region 38 in a case in which the determination unit 82C determines that the sizes 116 in the time series are stable is more prominent than the size-related information 44 displayed in the second display region 38 in a case in which the determination unit 82C determines that the sizes 116 in the time series are not stable. The change in the display aspect of the size-related information 44 is implemented by, for example, a change in font size, font color, and/or font brightness.

In this way, the display aspect of the size-related information 44 in the second display region 38 is changed in accordance with the determination result obtained by the determination unit 82C, so that the doctor 12 can easily ascertain whether or not the sizes 116 in the time series are stable.

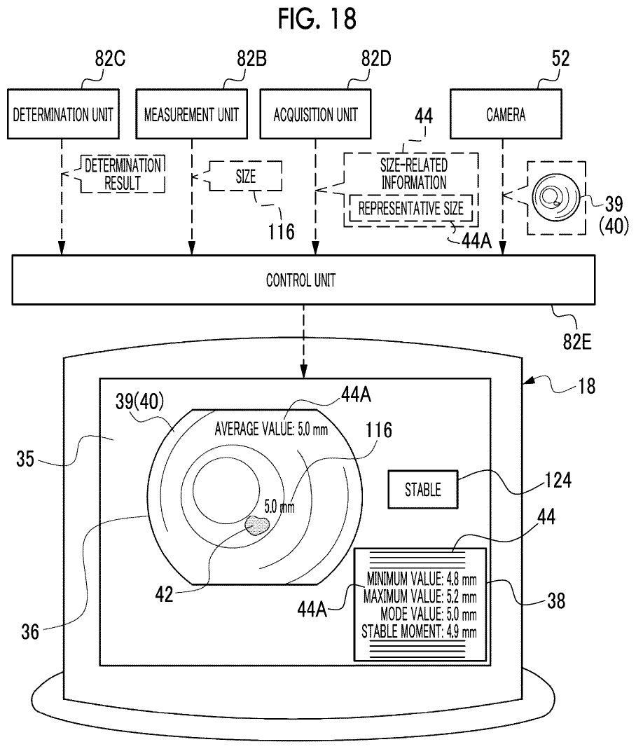

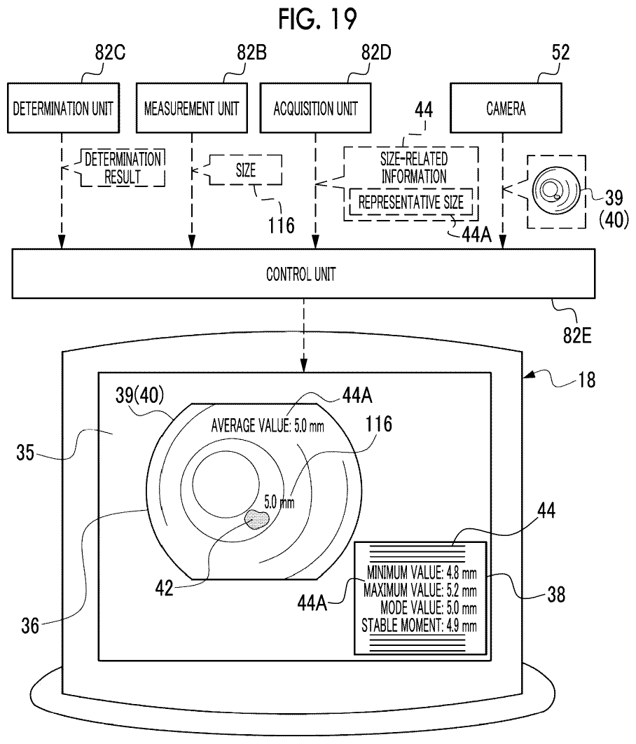

It should be noted that, in the examples shown in FIGS. 16 and 17, the form example has been described in which the size-related information 44 is displayed in the second display region 38, but at least a part of the size-related information 44 may be displayed in the first display region 36. For example, in the examples shown in FIGS. 18 and 19, the representative size 44A (here, for example, the average value) is displayed in the first display region 36. FIG. 18 shows an example in which the representative size 44A is displayed in the first display region 36 in a display aspect in a case in which the determination unit 82C determines that the sizes 116 in the time series are stable, and FIG. 19 shows an example in which the representative size 44A is displayed in the first display region 36 in a display aspect (display aspect that is less prominent than that in FIG. 18) in a case in which the determination unit 82C determines that the sizes 116 in the time series are not stable.

In addition, in the examples shown in FIGS. 16 and 17, the form example has been described in which the display aspect of the size-related information 44 in the second display region 38 is changed in accordance with the determination result obtained by the determination unit 82C, but the technology of the present disclosure is not limited to this. For example, the display aspect of the size 116 in the first display region 36 may be changed by the control unit 82E in accordance with the determination result obtained by the determination unit 82C. For example, the size 116 is displayed in bold characters in the first display region 36 as shown in FIG. 18 in a case in which the determination unit 82C determines that the sizes 116 in the time series are stable, and the size 116 is displayed in thin characters in the first display region 36 as shown in FIG. 19 in a case in which the determination unit 82C determines that the sizes 116 in the time series are not stable. This is merely an example, and the display aspect of the size 116 may be changed by the control unit 82E such that the size 116 displayed in the first display region 36 in a case in which the determination unit 82C determines that the sizes 116 in the time series are stable is more prominent than the size 116 displayed in the first display region 36 in a case in which the determination unit 82C determines that the sizes 116 in the time series are not stable. The change in the display aspect of the size 116 is implemented by, for example, a change in font size, font color, and/or font brightness. In this way, the display aspect of the size 116 in the first display region 36 is changed in accordance with the determination result obtained by the determination unit 82C, so that the doctor 12 can easily ascertain whether or not the sizes 116 in the time series are stable.

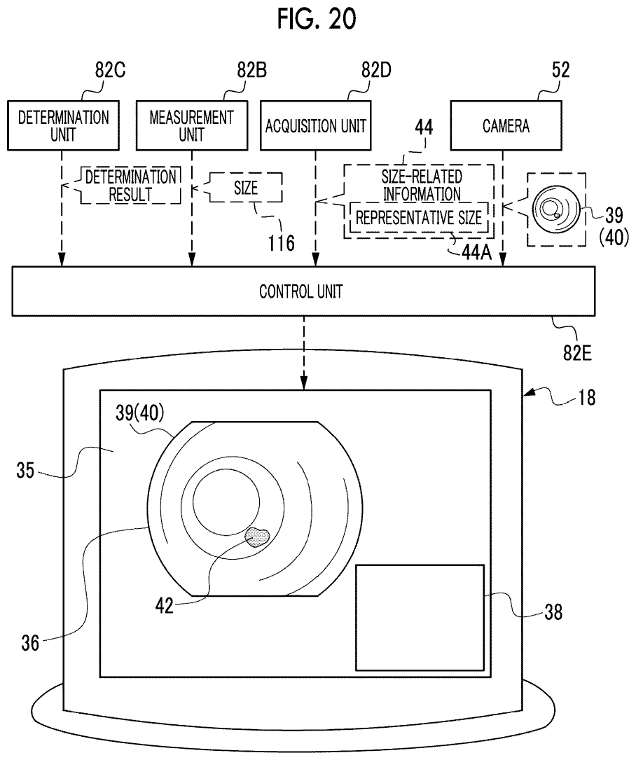

In the example shown in FIG. 19, the representative size 44A (here, for example, the average value) that is a part of the size 116 and the size-related information 44 displayed in the first display region 36 is displayed in a display aspect that is less prominent than the representative size 44A that is a part of the size 116 and the size-related information 44 shown in FIG. 18, but the technology of the present disclosure is not limited to this. For example, in a case in which the determination unit 82C determines that the sizes 116 in the time series are not stable, the size-related information 44 and the size 116 may not be displayed in the first display region 36 as shown in FIG. 20. In addition, in a case in which the determination unit 82C determines that the sizes 116 in the time series are not stable, the size-related information 44 or the size 116 may not be displayed in the first display region 36. In addition, in a case in which the determination unit 82C determines that the sizes 116 in the time series are not stable, for example, as shown in FIG. 20, the size-related information 44 may not be displayed in the second display region 38. As a result, the doctor 12 can easily ascertain whether or not the sizes 116 in the time series are stable.

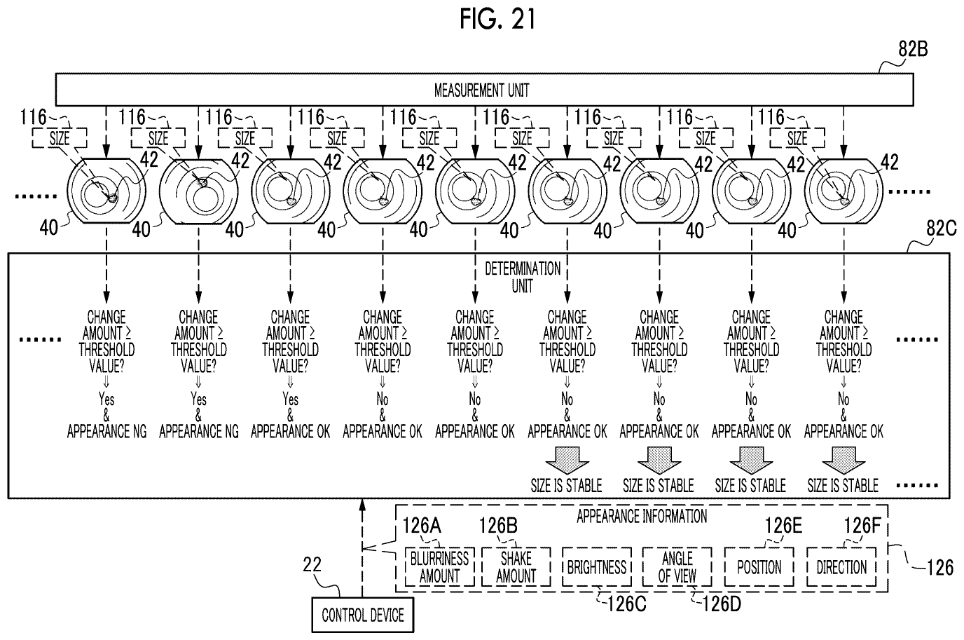

In the above-described embodiment, although the form example has been described in which the determination of whether or not the sizes 116 are stable is performed based on the size change amount, the technology of the present disclosure is not limited to this. For example, as shown in FIG. 21, the determination unit 82C may determine whether or not the sizes 116 are stable based on appearance information 126 in addition to the size change amount. For example, the appearance information 126 is acquired by the control device 22 and the like. The determination unit 82C acquires the appearance information 126 from the control device 22. The appearance information 126 is information indicating the appearance of the lesion 42 shown in the endoscopic video image 39. As the appearance information 126, a blurriness amount 126A of the endoscopic video image 39, a shake amount 126B of the camera 52, brightness 126C of the endoscopic video image 39, an angle of view 126D of the endoscopic video image 39, a position 126E of the lesion 42, which is shown in the endoscopic video image 39, in the endoscopic video image 39, and a direction 126F of an optical axis of the camera 52 with respect to a surface region (for example, a flat surface) including the lesion 42 (that is, an angle formed between the surface region including the lesion 42 and the optical axis of the camera 52) are used. It should be noted that, here, although the blurriness amount 126A, the shake amount 126B, the brightness 126C, the angle of view 126D, the position 126E, and the direction 126F have been described as examples, at least one or more of the blurriness amount 126A, the shake amount 126B, the brightness 126C, the angle of view 126D, the position 126E, or the direction 126F need only be used in the appearance information 126.

The determination unit 82C determines whether or not the appearance information 126 satisfies a predetermined condition (for example, a condition designated by the doctor 12 or the like). In a case in which the size change amount is less than the threshold value and the appearance information 126 satisfies the predetermined condition, the determination unit 82C determines that the sizes 116 in the time series are stable. In addition, in a case in which the appearance information 126 does not satisfy the predetermined condition regardless of whether or not the size change amount is less than the threshold value, the determination unit 82C determines that the sizes 116 in the time series are not stable.

Here, examples of the predetermined condition include a condition in which all of first to sixth conditions or at least one or more predetermined conditions (for example, one or more conditions designated in accordance with the given instruction and/or various conditions) are satisfied. Examples of the first condition include a condition in which the blurriness amount 126A is less than a predetermined blurriness amount. Examples of the second condition include a condition in which the shake amount 126B is less than a predetermined shake amount. Examples of the third condition include a condition in which the brightness 126C is less than predetermined brightness. Examples of the fourth condition include a condition in which the angle of view 126D is within a predetermined angle-of-view range. Examples of the fifth condition include a condition in which the position 126E is within a predetermined range (for example, a range other than a side edge portion (here, for example, a side edge portion affected by aberration of the lens of the camera 52) of the frame 40) in the frame 40. Examples of the sixth condition include a condition in which the direction 126F is a predetermined direction (for example, a direction in which the optical axis of the camera 52 is perpendicular to the surface region including the lesion 42 within an allowable error).

As described above, since it is determined whether or not the sizes 116 are stable based on the appearance information 126 in addition to the size change amount, and the blurriness amount 126A, the shake amount 126B, the brightness 126C, the angle of view 126D, the position 126E, and/or the direction 126F is used as the appearance information 126, the determination accuracy of whether or not the sizes 116 in the time series are stable is increased, so that the same or more effective effects as the effects of the above-described embodiment can be expected.

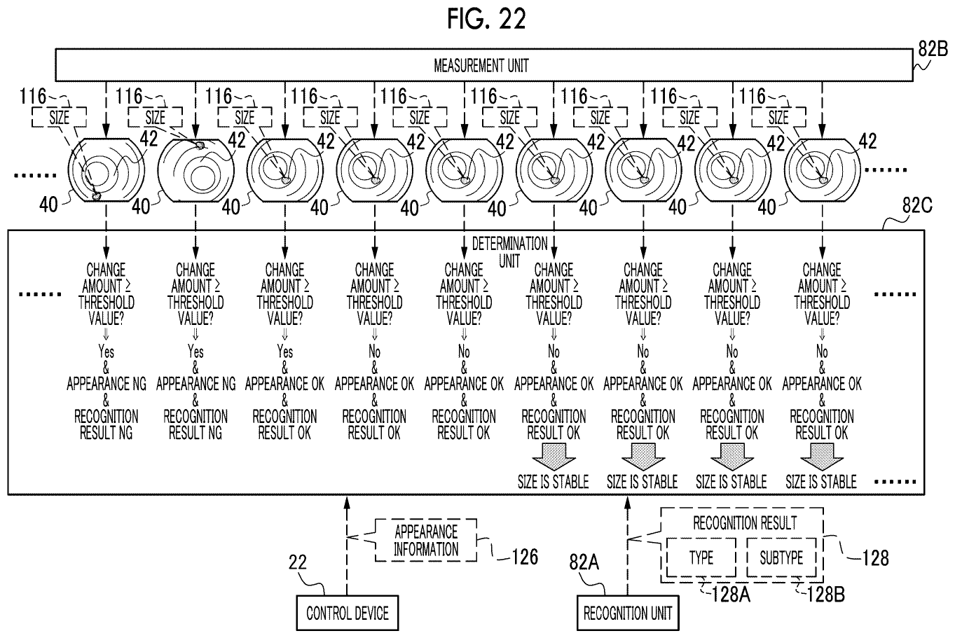

In addition, as shown in FIG. 22 as an example, the determination unit 82C may determine whether or not the sizes 116 are stable based on a recognition result 128, in addition to the size change amount and the appearance information 126. The recognition result 128 is a result of performing the recognition processing 96 on the endoscopic video image 39. The recognition result 128 includes a type 128A of the lesion 42 and/or a subtype 128B of the lesion 42.

The determination unit 82C determines whether or not the recognition result 128 satisfies a condition determined in advance (for example, a condition designated by the doctor 12 or the like). In a case in which the size change amount is less than the threshold value, the appearance information 126 satisfies the predetermined condition, and the recognition result 128 satisfies the predetermined condition, the determination unit 82C determines that the sizes 116 in the time series are stable. In a case in which the recognition result 128 does not satisfy the condition determined in advance, the determination unit 82C determines that the sizes 116 in the time series are not stable, regardless of whether or not the size change amount is less than the threshold value and whether or not the recognition result 128 satisfies the condition determined in advance.

As described above, by determining whether or not the sizes 116 are stable based on the recognition result 128 in addition to the size change amount and the appearance information 126, the determination accuracy of whether or not the sizes 116 in the time series are stable is increased, so that the same or more effective effects as the effects of the above-described embodiment can be expected.

It should be noted that, in the example shown in FIG. 22, the form example has been described in which it is determined whether or not the sizes 116 are stable based on the size change amount, the appearance information 126, and the recognition result 128, but it may be determined whether or not the sizes 116 are stable based on one or more of the size change amount, the appearance information 126, and the recognition result 128.

In the above-described embodiment, although the form example has been described in which the endoscopic video image 39 is displayed in the first display region 36, a result (for example, the recognition result 128) of the recognition processing 96 performed on the endoscopic video image 39 may be displayed superimposed on the endoscopic video image 39 in the first display region 36. In addition, at least a part of the segmentation image 102 obtained as a result of performing the recognition processing 96 on the endoscopic video image 39 may be displayed superimposed on the endoscopic video image 39. Examples of the form in which at least a part of the segmentation image 102 is displayed superimposed on the endoscopic video image 39 include a form example in which an outer contour of the segmentation image 102 is displayed superimposed on the endoscopic video image 39 using an alpha blending method.

In addition, for example, in a case in which the recognition processing 96 is performed by the AI-based bounding box method, the bounding box may be displayed superimposed on the endoscopic video image 39 in the first display region 36. In addition, for example, in a case in which a plurality of lesions 42 are shown in the endoscopic video image 39, at least a part of the segmentation image 102 and/or the bounding box may be displayed superimposed on the first display region 36 as information that can visually specify the lesion 42 corresponding to the measured size 116. In addition, the probability map 100 and/or the bounding box related to the lesion 42 corresponding to the measured size 116 may be displayed in a separate display region from the first display region 36. In addition, for example, the probability map 100 may be displayed superimposed on the endoscopic video image 39 in the first display region 36. The information displayed superimposed on the endoscopic video image 39 may be translucent information (for example, information rendered with alpha blending).

In the above-described embodiment, the form example has been described in which a length of a longest range in the real space that crosses the lesion 42 along the line segment 110 is measured as the size 116, but the technology of the present disclosure is not limited to this. For example, a length of a range corresponding to a longest line segment parallel to a short side of the circumscribing rectangular frame 112 with respect to the image region showing the lesion 42 in the real space may be measured as the size 116 and displayed on the screen 35. In this case, the doctor 12 can ascertain the length of the longest range in the real space that crosses the lesion 42 along the longest line segment parallel to the short side of the circumscribing rectangular frame 112 with respect to the image region showing the lesion 42.

In addition, the real size of the lesion 42 with respect to the radius and/or the diameter of the circumscribing circle for the image region indicating the lesion 42 may be measured and displayed on the screen 35. In this case, the doctor 12 can ascertain the real size of the lesion 42 with respect to the radius and/or the diameter of the circumscribing circle for the image region showing the lesion 42.

In the above-described embodiment, the form example has been described in which the size 116 is displayed in the first display region 36, but this is merely an example, and the size 116 may be displayed in a pop-up manner from the inside of the first display region 36 to the outside of the first display region 36, or the size 116 may be displayed in a region other than the first display region 36 on the screen 35. In addition, the type 128A and/or the subtype 128B may be displayed in the first display region 36 and/or the second display region 38, or may be displayed on a screen other than the screen 35.

In the above-described embodiment, the form example has been described in which the size of one lesion 42 is measured and the measurement result is presented to the doctor 12, but in a case in which a plurality of lesions 42 are shown in the endoscopic video image 39, the medical support processing may be executed for each of the plurality of lesions 42. In this case, a mark or the like may be added to the image region of the lesion 42 corresponding to the information displayed on the screen 35 such that it is possible to specify which information (size, subtype, type, and width) of the lesion 42 is displayed on the screen 35.

In the above-described embodiment, although the form example has been described in which the measurement of the size 116 is performed in units of one frame, this is merely an example, and the measurement of the size 116 may be performed in units of a plurality of frames.

In the above-described embodiment, the object recognition processing using the AI-based method has been described as the recognition processing 96, but the technology of the present disclosure is not limited to this, and the position of the lesion 42 shown in the endoscopic video image 39 may be recognized by the recognition unit 82A by executing object recognition processing using a non-AI-based method (for example, template matching or the like).

In the above-described embodiment, the form example has been described in which the arithmetic expression 114 is used to calculate the size 116, but the technology of the present disclosure is not limited to this, and the size 116 may be measured by performing AI-based processing on the frame 40. In this case, for example, in a case in which the frame 40 including the lesion 42 is input, a trained model that outputs the size 116 of the lesion 42 may be used. In a case of creating the trained model, a neural network may be trained through deep learning using training data in which an annotation indicating a size of a lesion is added as ground truth data to a lesion shown in an image used as example data.

In the above-described embodiment, the form example has been described in which the distance information 104 is derived by using the distance derivation model 94, but the technology of the present disclosure is not limited to this. For example, as another method of deriving the distance information 104 by using the AI-based method, for example, a method of combining segmentation and depth estimation (for example, regression learning of giving the distance information 104 to the entire image (for example, all pixels constituting the image) or unsupervised learning of learning the distance of the entire image in an unsupervised manner) is used.

In the above-described embodiment, the form example has been described in which the distance from the camera 52 to the intestinal wall 32 is derived by the AI-based method, but the distance from the camera 52 to the intestinal wall 32 may be actually measured. In this case, for example, a distance measurement sensor may be provided in the distal end portion 50 (see FIG. 2), and the distance from the camera 52 to the intestinal wall 32 may be measured by the distance measurement sensor.

In the above-described embodiment, the endoscopic video image 39 has been described as an example, but the technology of the present disclosure is not limited to this, and the technology of the present disclosure is established even in a medical video image other than the endoscopic video image 39 (for example, a video image obtained by a modality other than the endoscope system 10, such as a radiographic video image or an ultrasound video image).

In the above-described embodiment, although the form example has been described in which the size 116 of the lesion 42 shown in the video image is measured, this is merely an example, and the technology of the present disclosure is established even in a case where the size 116 of the lesion 42 shown in a frame-by-frame image or a still image is measured.

In the above-described embodiment, the form example has been described in which the distance information 104, which is extracted from the distance image 106, is input to the arithmetic expression 114, but the technology of the present disclosure is not limited to this. For example, the distance information 104 corresponding to the position specified from the position specifying information 98 may be extracted from all pieces of the distance information 104 output from the distance derivation model 94 without generating the distance image 106, and the extracted distance information 104 may be input to the arithmetic expression 114.

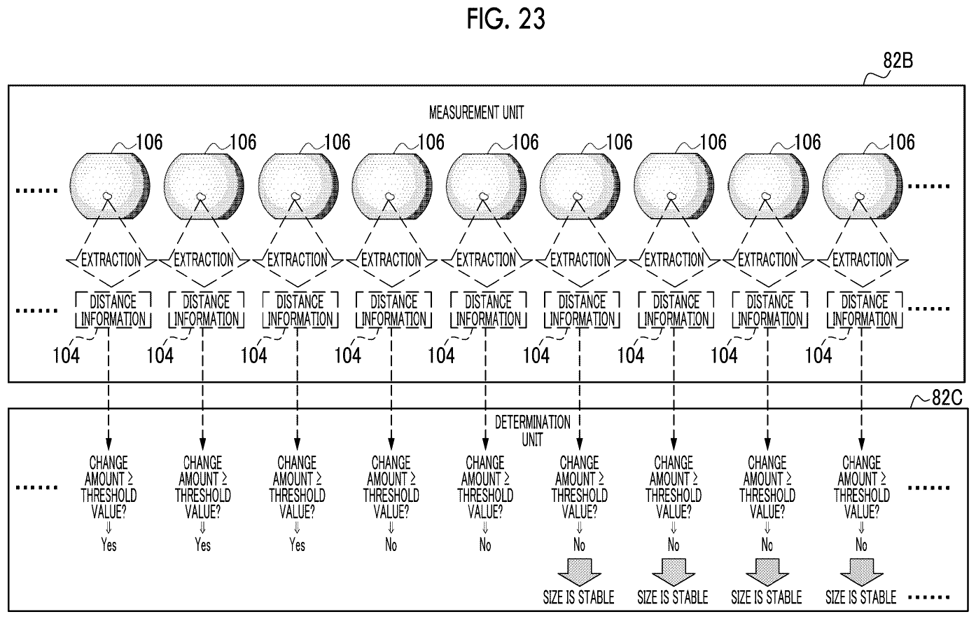

In the above-described embodiment, although the form example has been described in which the determination unit 82C determines whether or not the size change amount is equal to or greater than the threshold value, the technology of the present disclosure is not limited to this. For example, as shown in FIG. 23, the determination unit 82C may determine whether or not the change amount of the distance information 104 (see FIG. 6) extracted from the distance image 106 is equal to or greater than the threshold value. The change amount of the distance information 104 may be calculated by the measurement unit 82B or may be calculated by the determination unit 82C. The distance information 104 used for calculating the change amount may be extracted from the entire region of the distance image 106, or may be extracted from a line segment that crosses the distance image 106, or the distance information 104 (for example, a statistical value such as an average value, a median value, a mode value, a maximum value, or a minimum value among the distance information 104 included in the distance image 106) that is representative of all pieces of the distance information 104 included in the distance image 106 may be extracted from the distance image 106.

The determination unit 82C determines that the sizes 116 are not stable in a case in which the change amount of the distance information 104 extracted from the distance image 106 is equal to or greater than the threshold value, and the determination unit 82C determines that the sizes 116 are stable in a case in which the change amount of the distance information 104 extracted from the distance image 106 is less than the threshold value. In this manner, the same effects as the effects of the above-described embodiment can be expected.

It should be noted that the determination unit 82C may determine whether or not the sizes 116 in the time series are stable based on the determination result obtained by determining whether or not the size change amount is equal to or greater than the threshold value and the determination result obtained by determining whether or not the change amount of the distance information 104 extracted from the distance image 106 is equal to or greater than the threshold value. In this case, for example, in a case in which it is determined that the size change amount is less than the threshold value and it is determined that the change amount of the distance information 104 extracted from the distance image 106 is less than the threshold value, the determination unit 82C determines that the sizes 116 in the time series are stable.

In addition, the determination unit 82C may determine whether or not the sizes 116 in the time series are stable based on the appearance information 126 (see FIGS. 21 and 22) and/or the recognition result 128 (see FIG. 22), in addition to the determination result obtained by determining whether or not the size change amount is equal to or greater than the threshold value and the determination result obtained by determining whether or not the change amount of the distance information 104 extracted from the distance image 106 is equal to or greater than the threshold value.

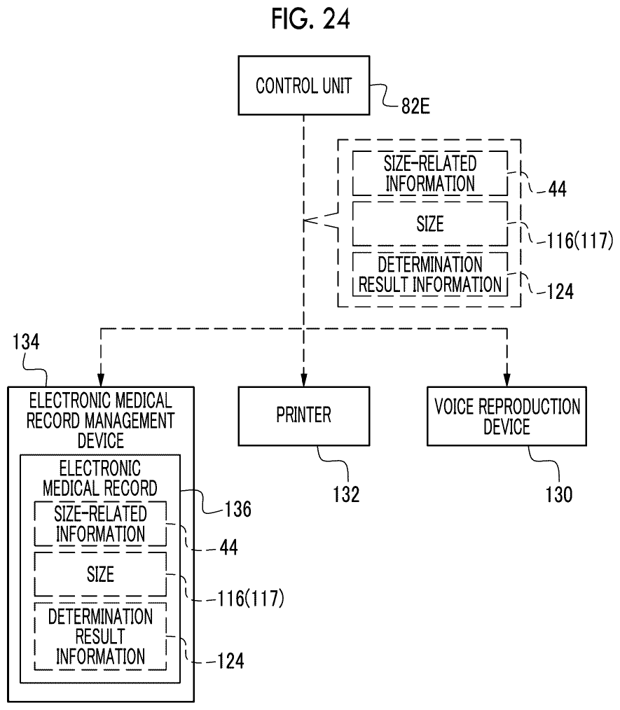

In the above-described example, the display device 18 has been described as the output destination of the size-related information 44, the sizes 116 and 117, the determination result information 124, and the like, but the technology of the present disclosure is not limited to this, and the output destination of various types of information (hereinafter, referred to as “various types of information”) such as the size-related information 44, the size 116, the size 117, and/or the determination result information 124 may be an output destination other than the display device 18. As shown in FIG. 24 as an example, the output destination of the various types of information includes a voice reproduction device 130, a printer 132, and/or an electronic medical record management device 134.

The various types of information may be output as voice by the voice reproduction device 130. In addition, the various types of information may be printed as text or the like on a medium (for example, paper) or the like by the printer 132. In addition, the various types of information may be stored in an electronic medical record 136 managed by the electronic medical record management device 134.

In the above-described example, although the form example has been described in which various types of information are displayed on the screen 35 or various types of information are not displayed on the screen 35, the display of various types of information on the screen 35 means display of various types of information in a state of being perceivable by the user (for example, the doctor 12). Furthermore, the concept that various types of information are not displayed on the screen 35 also includes the concept that a display level of various types of information is lowered (for example, a level at which various types of information are perceived by the display). For example, the concept that various types of information are not displayed on the screen 35 also includes the concept that various types of information are displayed in a display aspect in which the various types of information are not visually perceived by the user and the like. Examples of the display aspect in this case include a display aspect in which various types of information are reduced in font size, various types of information are thinned, various types of information are dotted, various types of information are made to blink, various types of information are displayed for an imperceptible display time, or various types of information are displayed to be transparent to an imperceptible level. It should be noted that the same applies to various types of output such as the above-described sound output, printing, and storage.

In the above-described embodiment, the form example has been described in which the medical support processing is executed by the processor 82 included in the endoscope system 10, but the technology of the present disclosure is not limited to this, and the device that executes at least a part of processing included in the medical support processing may be provided outside the endoscope system 10.

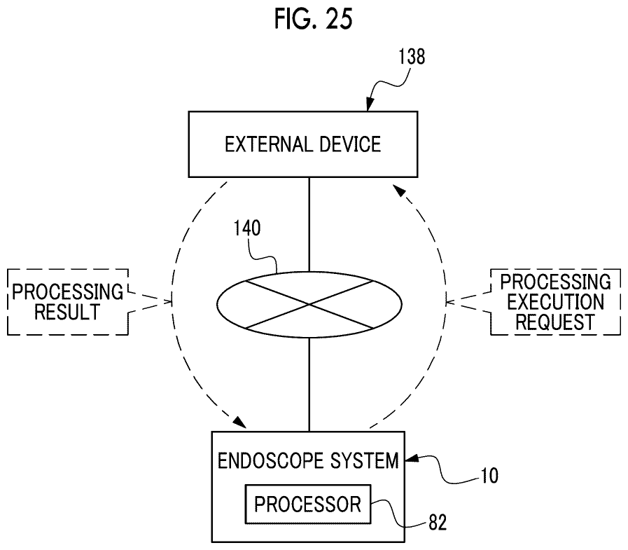

In this case, for example, as shown in FIG. 25, an external device 138, which is communicably connected to the endoscope system 10 via a network 140 (for example, a WAN and/or a LAN), may be used.

Examples of the external device 138 include at least one server that directly or indirectly transmits and receives data to and from the endoscope system 10 via the network 140. The external device 138 receives a processing execution instruction given from the processor 82 of the endoscope system 10 via the network 140. Then, the external device 138 executes processing in accordance with the received processing execution instruction, and transmits a processing result to the endoscope system 10 via the network 140. In the endoscope system 10, the processor 82 receives the processing result transmitted from the external device 138 via the network 140, and executes the processing using the received processing result.

Examples of the processing execution instruction include an instruction for the external device 138 to execute at least a part of the medical support processing. Examples of at least a part of the medical support processing (that is, the processing to be executed by the external device 138) include processing executed by the recognition unit 82A, processing executed by the measurement unit 82B, processing executed by the determination unit 82C, processing executed by the acquisition unit 82D, and/or processing executed by the control unit 82E.

The external device 138 is implemented by, for example, cloud computing. The cloud computing is merely an example, and the external device 138 may be implemented by network computing, such as fog computing, edge computing, or grid computing. At least one personal computer or the like may be used as the external device 138, instead of the server. In addition, an operation device having a communication function equipped with a plurality of types of AI functions may be used.

In the above-described embodiment, the form example has been described in which the NVM 86 stores the medical support program 90, but the technology of the present disclosure is not limited to this. For example, the medical support program 90 may be stored in a portable non-transitory computer-readable storage medium such as an SSD or a USB memory. The medical support program 90, which is stored in the non-transitory storage medium, is installed in the computer 78 of the endoscope system 10. The processor 82 executes the medical support processing in accordance with the medical support program 90.

In addition, the medical support program 90 may be stored in a storage device of another computer, a server, or the like that is connected to the endoscope system 10 via the network, and the medical support program 90 may be downloaded and installed in the computer 78 in response to a request from the endoscope system 10.

It should be noted that it is not necessary to store the entire medical support program 90 in a storage device of another computer or server device connected to the endoscope system 10 or to store the entire medical support program 90 in the NVM 86, and a part of the medical support program 90 may be stored.

The following various processors can be used as hardware resources for executing the medical support processing. An example of the processor is a CPU which is a general-purpose processor that executes software, that is, a program, to function as the hardware resource for executing the medical support processing. In addition, an example of the processor is a dedicated electric circuit which is a processor having a dedicated circuit configuration designed to execute specific processing, such as an FPGA, a PLD, or an ASIC. All processors have a memory built therein or connected thereto, and all processors use the memory to execute the medical support processing.

The hardware resource for executing the medical support processing may be configured by one of the various processors or by a combination of two or more processors of the same type or different types (for example, a combination of a plurality of FPGAs or a combination of a CPU and an FPGA). Further, the hardware resource for executing the medical support processing may be one processor.

A first example of the configuration in which the hardware resource is configured by one processor is an aspect in which one processor is configured by a combination of one or more CPUs and software, and this processor functions as the hardware resource for executing the medical support processing. As a second example, as typified by an SoC or the like, there is a form in which a processor that implements all functions of a system including a plurality of hardware resources executing the medical support processing with one IC chip is used. As described above, the medical support processing is implemented by using one or more of the various processors as the hardware resource.

Further, as the hardware structure of the various processors, specifically, an electronic circuit in which circuit elements, such as semiconductor elements, are combined can be used. Further, the above-described medical support processing is merely an example. Therefore, it goes without saying that unnecessary steps may be deleted, new steps may be added, or the processing order may be changed, within a range that does not deviate from the gist of the present disclosure.

The above-described contents and the above-shown contents are detailed descriptions of portions relating to the technology of the present disclosure and are merely examples of the technology of the present disclosure. For example, the description of the configuration, the function, the operation, and the effect above are the description of examples of the configuration, the function, the operation, and the effect of the parts according to the technology of the present disclosure. As a result, it goes without saying that unnecessary parts may be deleted, new elements may be added, or replacements may be made with respect to the above-described contents and the above-shown contents within a range that does not deviate from the gist of the technology of the present disclosure. Further, the description of, for example, common technical knowledge that does not need to be particularly described to enable the implementation of the technology of the present disclosure is omitted in the above-described contents and the above-shown contents in order to avoid confusion and to facilitate understanding of the portions relating to the technology of the present disclosure.

In the present specification, “A and/or B” is synonymous with “at least one of A or B”. That is, “A and/or B” may mean only A, only B, or a combination of A and B. In addition, in the present specification, the same concept as “A and/or B” is applied to a case in which the connection of three or more matters is expressed by “and/or”.

All of the documents, the patent applications, and the technical standards described in the present specification are incorporated into the present specification by reference to the same extent as in a case in which each of the documents, the patent applications, and the technical standards are specifically and individually stated to be described by reference.

What is claimed is:

1. A medical support device comprising:
a processor,
wherein the processor is configured to:
acquire size-related information that is information corresponding to sizes in a time series of an observation target region shown in a medical video image; and
output the size-related information, and
a representative value of the sizes in the time series is used as the size-related information.

2. The medical support device according to claim 1,
wherein the representative value is a value that is representative of the sizes measured in the time series based on a plurality of frames included in a first period of the medical video image.

3. The medical support device according to claim 2,
wherein the representative value includes a maximum value of the sizes within the first period, a minimum value of the sizes within the first period, a frequency of the sizes within the first period, an average value of the sizes within the first period, a median value of the sizes within the first period, and/or a variance value of the sizes within the first period.

4. The medical support device according to claim 2,
wherein the representative value includes a frequency of the sizes within the first period, and
a histogram of the frequency is used as the size-related information.

5. The medical support device according to claim 2,
wherein the representative value includes a maximum value and a minimum value of the size within the first period, and
fluctuation range information indicating a fluctuation range from the maximum value to the minimum value is used as the size-related information.

6. The medical support device according to claim 1,
wherein the processor is configured to acquire the size-related information in a case in which the sizes in the time series are stable.

7. The medical support device according to claim 6,
wherein the processor is configured to:
output the size-related information in a case in which the sizes in the time series are stable; and
not output the size-related information in a case in which the sizes in the time series are not stable.

8. The medical support device according to claim 6,
wherein the processor is configured to:
output the sizes in a case in which the sizes in the time series are stable; and
not output the sizes in a case in which the sizes in the time series are not stable.

9. The medical support device according to claim 6,
wherein whether or not the sizes in the time series are stable is determined based on a recognition result of the observation target region, measurement results of the sizes, and/or an appearance of the observation target region shown in the medical video image.

10. The medical support device according to claim 9,
wherein, in a case in which a change amount of the sizes in the time series within a second period and/or a change amount of distance information included in a distance image for the observation target region is less than a threshold value,
it is determined that the sizes in the time series are stable.

11. The medical support device according to claim 10,
wherein the observation target region is recognized by a method using AI,
the change amount of the sizes is a change amount of a closed region that defines the observation target region recognized by the method using the AI,
the closed region is a bounding box or a segmentation image obtained from the AI, and
the change amount of the distance information is a change amount of the distance information included in the distance image corresponding to the closed region.

12. The medical support device according to claim 9,
wherein the appearance includes a blurriness amount, a shake amount, brightness, an angle of view, a position, and/or a direction.

13. The medical support device according to claim 9,
wherein the processor is configured to output determination result information indicating whether or not the sizes in the time series are stable.

14. The medical support device according to claim 1,
wherein the output of the size-related information is implemented by displaying the size-related information on a first screen.

15. The medical support device according to claim 14,
wherein the processor is configured to:
selectively display, on the first screen, temporal change information that enables specification of a temporal change of the sizes in the time series and the size-related information; and
in a case in which the sizes in the time series are stable in a state in which the temporal change information is displayed on the first screen, switch information displayed on the first screen from the temporal change information to the size-related information.

16. The medical support device according to claim 14,
wherein the processor is configured to change a display aspect of the size-related information on the first screen in accordance with whether or not the sizes in the time series are stable.

17. The medical support device according to claim 1,
wherein the processor is configured to:
display the sizes in the time series on a second screen; and
change a display aspect of the sizes on the second screen in accordance with whether or not the sizes in the time series are stable.

18. The medical support device according to claim 1,
wherein the processor is configured to display the sizes in the time series on a third screen,
the size displayed on the third screen is a real number represented by a plurality of digits, and
a font size, a font color, and/or font brightness of the real number is changed in units of the digits.

19. The medical support device according to claim 1,
wherein the processor is configured to:
display a recognition result of the observation target region and/or measurement results of the sizes in a state of being superimposed on the medical video image; and
display the size-related information in a separate display region from the medical video image.

20. The medical support device according to claim 1,
wherein the medical video image is an endoscopic video image obtained by imaging with an endoscope.

21. The medical support device according to claim 1,
wherein the observation target region is a lesion.

22. An endoscope system comprising:
the medical support device according to claim 1; and
an endoscope that is inserted into a body including the observation target region and that images the observation target region to acquire the medical video image.

23. A medical support method comprising:
acquiring size-related information that is information corresponding to sizes in a time series of an observation target region shown in a medical video image; and
outputting the size-related information,
wherein a representative value of the sizes in the time series is used as the size-related information.

24. The medical support method according to claim 23, further comprising:
using an endoscope that performs imaging to acquire the medical video image.

25. A non-transitory computer-readable storage medium storing a program executable by a computer to execute medical support processing comprising:
acquiring size-related information that is information corresponding to sizes in a time series of an observation target region shown in a medical video image; and
outputting the size-related information,
wherein a representative value of the sizes in the time series is used as the size-related information.
